(19) United States
(12) Patent Application Publication
(10) Pub. No.: US 2022/0338463 A1
(43) Pub. Date: Oct. 27, 2022

(54) METHOD OF PRODUCING LYOPHILIZED CELLS

(71) Applicant: LONZA LTD, Visp (CH)

(72) Inventors: Dhananjay JERE, Basel (CH); Hanns-Christian MAHLER, Lörrach (DE); Susanne JÖRG, Basel (CH); Gregor BORDON, Basel (CH)

(21) Appl. No.: 17/641,376

(22) PCT Filed: Sep. 11, 2020
(86) PCT No.: PCT/US2020/050434

§ 371 Date: Mar. 8, 2022

Related U.S. Application Data
(60) Provisional application No. 62/900,184, filed on Sep. 13, 2019.
(60) Provisional application No. 62/904,490, filed on Sep. 23, 2019.

(51) Int. Cl. A01N 1/02; C12N 5/0775; C12N 5/074; C12N 5/09

(52) U.S. Cl. CPC A01N 1/0221; A01N 1/0278; C12N 5/0662; C12N 5/0696; C12N 5/0693; C12N 2533/54

(57) ABSTRACT

The present disclosure provides a method of producing a population of lyophilized cells, comprising: (a) freezing a composition comprising a population of cells, an aqueous component, a polyol, a sugar, and a polysaccharide; and (b) removing at least 90% of the aqueous component from the frozen composition to produce the population of lyophilized cells. On some embodiments, the disclosure provides a method of producing a population of reconstituted viable cells, comprising: (a) freezing a composition comprising a population of cells, an aqueous component, a polyol, a sugar, and a polysaccharide; (b) removing at least 90% of the aqueous component from the frozen composition to produce the population of lyophilized cells, and (c) resuspending the population of lyophilized cells in a reconstitution agent to form a reconstituted composition, wherein at least 1% of the cells are viable.

### FIELD OF THE INVENTION

The present disclosure provides a method of producing a population of lyophilized cells, comprising: (a) freezing a composition comprising a population of cells, an aqueous component, a polyol, a sugar, and a polysaccharide; and (b) removing at least 90% of the aqueous component from the frozen composition to produce the population of lyophilized cells. In some embodiments, the disclosure provides a method of producing a population of reconstituted viable cells, comprising: (a) freezing a composition comprising a population of cells, an aqueous component, a polyol, a sugar, and a polysaccharide; (b) removing at least 90% of the aqueous component from the frozen composition to produce the population of lyophilized cells, and (c) resuspending the population of lyophilized cells in a reconstitution agent to form a reconstituted composition, wherein at least 1% of the cells are viable.

### BACKGROUND

Cellular therapy, also referred to as also live cell therapy, cellular suspensions, glandular therapy, stem and embryonic cell therapy, refers to various procedures in which processed tissue from cell culture, embryos, fetuses or organs, is injected, infused or administered by other parenteral routes, or taken orally, nasally or by inhalation for the treatment of a condition or disease. Cellular therapy products (CTPs), also known as cell based medicinal products (CBMPs, include cellular immunotherapies, cancer vaccines, and other types of both autologous and allogeneic cells for certain therapeutic indications, including hematopoietic stem cells and adult and embryonic stem cells.

Cell therapies are able to sense diverse signals, move to specific parts of the body, gather inputs from their environment and execute appropriate responses. CTPs can be classified according to their therapeutic indication, cell type (most common) or technology used to prepare them. Classifications include (i) somatic cell technologies (Weissman, I. L., Science 287, 1442-1446 (2000).), (ii) Cell immortalisation techniques (Pollock, K. et al., Exp. Neurol. 199, 143-155 (2006); Carter, M. & Shieh, J. Chapter 14—Cell Culture Techniques. in Guide to Research Techniques in Neuroscience (Second Edition) (eds. Carter, M. & Shieh, J.) 295-310 (Academic Press, 2015)), (iii) Ex vivo gene modification of cells using viral vectors such as Chimeric antigen receptor T-cell (CAR-T) therapy, (iv) In vivo gene modification of cells using viral vectors (M. Kay, Nature Reviews Genetics volume 12, pages 316-328 (2011)), (v) Genome editing technologies such as CRISPR—Cas9 technology an example of which stem cells of a patient can be removed, expanded, genetically modify by using the CRISPR—Cas9 technology and transfused back into the patient, and (vi) Cell plasticity technologies (Takahashi, K. et al. Induction of pluripotent stem cells from adult human fibroblasts by defined factors. Cell 131,861-872 (2007)).

In order to bring CTPs to market, the CTPs must be produced on a larger scale than in the laboratory. In some instances, the cells must be transported and stored for long periods of time. Current storage techniques include cryopreservation, chilled storage, and ambient temperature pausing.

In cryopreservation, chemical and metabolic processes inside the cells are stopped by freezing the intracellular water. While the post-thaw viability of the cells is cell line dependent, usually the cryopreservation is considered to be successful when more than 90% of the cells survive the procedure. See, e.g., Jesson, H. E., et al., Chapter 11—Storage and Delivery of Stem Cells for Cellular Therapies. in Stem Cell Manufacturing (eds. Cabral, J. M. S., Lobato de Silva, C., Chase, L. G. & Margarida Diogo, M.) 233-264 (Elsevier, 2016).

Common cryopreservation practice involves slowly freezing the cells, e.g., at −80° C., and placing them into liquid nitrogen at −196° C. or liquid nitrogen vapor phase −150° C. afterwards. Care has to be taken when choosing the cooling rate. In case of slow cooling, a hypertonic solution is generated in the extracellular unfrozen water fraction, which may expose the cells to a high osmotic stress, resulting in dehydration and shrinkage. In case of rapid cooling, there is no time for dehydration and ice crystals are formed intracellularly, which causes injuries. Freezing, and subsequent thawing of the frozen cells has a major impact on cell viability. Most usually, the CTP is shipped to the clinical facility frozen and then thawed and administered. Complicated thawing processes can result in inconsistencies and variability in the quality of the final product. See, e.g., Zhang, M., et al., Biochimica et Biophysica Acta (BBA)—Biomembranes 1858: 1400-1409 (2016), and Campbell, A. et al. Concise Review: Process Development Considerations for Cell Therapy. Stem Cells Transl Med 4: 1155-1163 (2015). Further potential problems associated with cryopreservation include the risk of container integrity breach, increasing the risk of insterility and raising patient safety concerns. Additionally, the quality of the cells usually after thawing has to be verified due to inconsistencies raised in the thawing process.

In addition to choosing a correct cooling rate, the addition of cryoprotective agents can be imperative for cell survival. Dimethylsulfoxide (DMSO) is commonly used, which is present in most CTP formulations at concentrations of 2-10%. Although DMSO is an often used cryopreserving agent, concerns regarding its toxicity exists since often CTPs are administered to more delicate parts of the body. This is especially true for CTPs administered to the central nervous system.

Alternative cryoprotectants, such as glycerol and several sugars have also been tested, but none appear to work as well as DMSO. A possible reason for sugars not being as successful cryoprotectants is that sugars do not pass the cell membrane, in contrast to DMSO and glycerol. See, e.g., Jesson, H. E., 2016, supra and Zhang, M., 2015, supra.

Cryopreservation, e.g., preservation in liquid nitrogen or in frozen state below about −20° C., is a standard for long-term storage. However, cryopreservation brings many logistical and supply chain challenges when shipping the therapeutics across the world. For temporary storage, it is possible to utilize chilled storage, i.e., have the cells preserved at 2-8° C. For example, human mesenchymal stem cells (hMSCs) can be successfully stored for up to 96 hours. See, Ginis, I., et al., Tissue Eng Part C Methods 18: 453-463 (2012). Many other cell types survive even shorter time under hypothermic conditions, which limits the use of chilled storage.

Storage at ambient temperature, i.e., ambient temperature pausing, has been used in limited circumstanced. Two main techniques are used for pausing the cells at ambient temperature: alginate gel entrapment and enclosure in hermetic culture chambers. These systems are meant to protect the cells from cold-induced stress and mild hypoxia, which is employed to stop the cells in a pre-mitosis phase where they are not actively dividing. Jesson, 2016, supra.

Like chilled storage, ambient temperature pausing is appropriate only for temporary storage. Example of possible usage of such condition is pooling of cells during the production process, e.g. after harvest, when the cells are waiting for down-stream processing for instance. One main challenge associated with ambient temperature pausing is that the principle has not yet been proven the on industrial scale.

### SUMMARY OF THE INVENTION

The present disclosure is directed to a method of lyophilizing a population of cells, wherein the cells are stable, and can remain viable at increased temperatures. In some embodiments, the disclosure provide a method of producing a population of lyophilized cells, comprising: (a) freezing a composition comprising a population of cells, an aqueous component, a polyol, a sugar, and a polysaccharide; and (b) removing at least about 90% of the aqueous component from the frozen composition to produce the population of lyophilized cells.

In some embodiments, the disclosure provides a method of producing a population of reconstituted viable cells, comprising: (a) freezing a composition comprising a population of cells, an aqueous component, a polyol, a sugar, and a polysaccharide; (b) removing at least about 90% of the aqueous component from the frozen composition to produce the population of lyophilized cells, and (c) resuspending the population of lyophilized cells in a reconstitution agent to form a reconstituted composition, wherein at least about 1% of the cells are viable.

In some embodiments, the composition of (a) further comprises a hydrogel. In some embodiments, the hydrogel is a biocompatible hydrogel. In some embodiments, the hydrogel is a hyaluronan gel, alginate gel, agarose gel, collagen gel or combination of thereof. In some embodiments, the population of cells in (a) is suspended in the hydrogel.

In some embodiments, the freezing of (a) is performed either in suspension or attached in a container. In some embodiments, the freezing of (a) is performed with the population of cells suspended in a hydrogel. In some embodiments, the lyophilization is performed in a container with or without collagen coating. In some embodiments, the container is a glass or plastic container without or without membrane for cell attachment or growth.

In some embodiments, the population of cells of (a) is about 1×10⁴ cells per mL to 1×10⁶ cells per mL. In some embodiments, the population of cells of (a) is about 1×10⁵ cells per mL to 4×10⁵ cells per mL. In some embodiments, the population of cells of (a) is about 2×10⁵ cells per mL to 2.5×10⁵ cells per mL.

In some embodiments, the sugar is a disaccharide. In some embodiments, the disaccharide is selected from the group consisting of maltose, lactose, sucrose, lactulose, trehalose, cellobiose, isomaltose, melibiose, and gentiobiose. In some embodiments, the disaccharide has a C₁-C₁ glycosidic linkage. In some embodiments, the sugar is trehalose.

In some embodiments, prior to the freezing in (a), the population of cells is isolated and resuspended in a poration solution. In some embodiments, the poration solution comprises sugar and water. In some embodiments, the sugar in the poration solution is about 0.1 M to about 1.0M. In some embodiments, the poration solution comprises trehalose and water. In some embodiments, the trehalose in the poration solution is about 0.1 M to about 1.0M. In some embodiments, the trehalose in the poration solution is about 0.2 M to about 0.6 M. In some embodiments, the trehalose in the poration solution is about 0.4 M.

In some embodiments, the polyol is selected from glycerol, propylene glycol and ethylene glycol. In some embodiments, the polyol is about 0.05 M to about 5 M.

The method of any one of claims 1 to 26, wherein the polyol is about 0.1 M to about 3 M. In some embodiments, the polyol is about 0.5 M to about 2 M. In some embodiments, the polyol is about 1 M to about 1.5 M.

In some embodiments, the polyol is glycerol. In some embodiments, the glycerol is about 0.01 M to about 10 M in the composition. In some embodiments, the glycerol is about 0.05 M to about 5 M. In some embodiments, the glycerol is about 0.1 M to about 3 M. In some embodiments, the glycerol is about 0.5 M to about 2 M. In some embodiments, the glycerol is about 1 M to about 1.5 M.

In some embodiments, the polysaccharide is about 1 mg/mL to about 25 mg/mL. In some embodiments, the polysaccharide is about 5 mg/mL to about 15 mg/mL. In some embodiments, the polysaccharide is about 10 mg/mL. In some embodiments, the polysaccharide is a polyglucan. In some embodiments, the polyglucan is a dextran. In some embodiments, the dextran has a molecular weight of about 10,000 Da to about 60,000 Da. In some embodiments, the dextran has a molecular weight of about 30,000 Da to about 50,000 Da. In some embodiments, the dextran has a molecular weight of about 40,000 Da.

In some embodiments, the composition of (a) is free of DMSO. In some embodiments, the composition comprising the population of cells in (a) comprises thiobarbituric acid, polyvinylpyrrolidone, polyoxyethylene stearyl ether, polyethylene glycol, cyclodextrin, hydroxyethyl starch, or combinations thereof.

In some embodiments, the reconstitution agent comprises polyvinylpyrrolidone, trehalose, sucrose, glucose or combinations thereof. In some embodiments, the reconstitution agent comprises polyvinylpyrrolidone. In some embodiments, the reconstitution agent comprises a phosphate buffer solution. In some embodiments, the reconstitution agent comprises water for injection.

In some embodiments, the composition is an isotonic solution. In some embodiments, the composition is a hypotonic solution. In some embodiments, the composition is a hypertonic solution.

In some embodiments, the cell is a mammalian cell. In some embodiments, the cell is a stem cell. In some embodiments, the cell is a pluripotent stem cell, an embryonic stem cell, a mesenchymal stem cell, or a hematopoietic stem cell. In some embodiments, the cell is a mesenchymal stem cell. In some embodiments, the cell is an induced pluripotent stem cell. In some embodiments, the population of cells are neuroblastoma cells. In some embodiments, the neuroblastoma cells are SK-N-AS cells.

In some embodiments, the aqueous component comprises buffer. In some embodiments, the buffer comprises phosphate buffer, Tris buffer, acetate buffer, bicarbonate buffer, histidine buffer, citrate buffer or combinations thereof. In some embodiments, the aqueous component comprises cell culture medium. In some embodiments, the cell culture medium is free of serum.

In some embodiments, the removing the aqueous component comprises lowering pressure, applying heat, or both, to the frozen composition to remove the aqueous component. In some embodiments, the removing the aqueous component comprises a primary drying step and a secondary drying step. In some embodiments, the removing the aqueous component comprises greater than two drying steps. In some embodiments, the primary drying step comprises lowering pressure to remove the aqueous component. In some embodiments, the secondary drying step comprises applying heat to remove the aqueous component.

In some embodiments, the freezing occurs at between about −10° C. to about −100° C. In some embodiments, the freezing occurs at between about −20° C. to about −90° C. In some embodiments, the freezing occurs at between about −40° C. to about −60° C. In some embodiments, the freezing of (a) lowers the temperature of the composition to about −50° C. In some embodiments, the freezing of (a) lowers the temperature of the composition to about −50° C., the aqueous component is removed at a pressure of a chamber pressure of about 20 mTorr to about 40 mTorr.

In some embodiments, the resuspending the lyophilized cell occurs greater than two hours after the removing the aqueous component. In some embodiments, the resuspending the lyophilized cells occurs greater than one day after the removing the aqueous component. In some embodiments, the resuspending the lyophilized cells occurs greater than one week after the removing the aqueous component. In some embodiments, the resuspending the lyophilized cells occurs greater than one month after the removing the aqueous component. In some embodiments, the lyophilized cells are stored below about −20° C. prior to the resuspending. In some embodiments, the lyophilized cells are stored at about −20° C. to about 30° C. prior to the resuspending. In some embodiments, the lyophilized cells are stored at about 4° C. to about 28° C. prior to the resuspending. In some embodiments, the lyophilized cells are stored at about 10° C. to about 27° C. prior to the resuspending. In some embodiments, the lyophilized cells are stored at about 15° C. to about 26° C. prior to the resuspending. In some embodiments, the lyophilized cells are stored at about 20° C. to about 25° C. prior to the resuspending. In some embodiments, the lyophilized cells are stored at about 0° C. to about 8° C. prior to the resuspending. In some embodiments, the lyophilized cells are stored at greater than about −20° C. for greater than 2 days prior to the resuspending. In some embodiments, the lyophilized cells are stored at about 20° C. to about 25° C. for greater than 1 week prior to the resuspending. In some embodiments, the lyophilized cells are stored at about 2° C. to about 8° C. for greater than 1 week prior to the resuspending.

In some embodiments, at least about 20% of the cells in the reconstituted composition are viable. In some embodiments, at least about 30% of the cells in the reconstituted composition are viable. In some embodiments, at least about 40% of the cells in the reconstituted composition are viable. In some embodiments, at least about 50% of the cells in the reconstituted composition attach as measured by ALAMARBLUE® test.

In some embodiments, the disclosure provides a composition comprising a population of cells, an aqueous component, a polyol, a sugar, and polysaccharide, wherein the composition is less than about 10% (vol/vol) water, and wherein the population of cells can be resuspended, wherein at least about 1% the cells in the population of cells are viable when resuspended.

In some embodiments, the disclosure provides a composition comprising a population of cells, an aqueous component, glycerol, trehalose, and polysaccharide, wherein the composition is less than about 10% (vol/vol) water, and wherein the population of cells can be resuspended, wherein at least about 1% the cells in the population of cells are viable when resuspended.

In some embodiments, the disclosure provides a composition comprising a population of cells, an aqueous component, about 0.5 M to about 2 M polyol, about 0.2 M to about 0.6 M sugar, and about 2 mg/mL to about 20 mg/mL polysaccharide, wherein the population of cells is about 2×10⁵ cell/cm² to about 3×10⁵ cells/cm².

In some embodiments, the disclosure provides a composition comprising a population of cells, an aqueous component, about 0.5 M to about 2 M glycerol, about 0.2 M to about 0.6 M trehalose, and about 2 mg/mL to about 20 mg/mL polysaccharide, wherein the population of cells are about 2×10⁵ cell/cm² to about 3×10⁵ cells/cm². In some embodiments, the aqueous component in the population of lyophilized cells is less than about 5% w/w.

In some embodiments, the disclosure provides a method of lyophilizing a population of cells, comprising: (a) freezing a composition comprising the population of cells, an aqueous component, about 0.5 M to about 2 M glycerol, about 0.2 M to about 0.6 M trehalose, and about 2 mg/mL to about 20 mg/mL polysaccharide, wherein the freezing occurs below about −30° C.; and (b) removing the aqueous component from the frozen composition to produce a population of lyophilized cells, wherein the aqueous component in the population of lyophilized cells is less than about 5% w/w. In some embodiments, the composition of (a) further comprises a hydrogel. In some embodiments, the hydrogel is a hyaluronan gel, alginate gel or collagen gel. In some embodiments, the population of cells in (a) is suspended in the hydrogel.

In some embodiments, the disclosure provides a method of producing viable population of cells, comprising: (a) freezing a composition comprising the population of cells, an aqueous component, about 0.5 M to about 2 M polyol, about 0.2 M to about 0.6 M sugar, and about 2 mg/mL to about 20 mg/mL polysaccharide, wherein the freezing occurs below about −30° C.; (b) removing the aqueous component from the frozen composition to produce a population of lyophilized cells, wherein the aqueous component in the population of lyophilized cells is less than about 5% w/w; and (c) resuspending the population of lyophilized cells in a reconstitution agent to form a reconstituted composition, wherein at least about 5% of the cells in the reconstituted composition are viable.

In some embodiments, the disclosure provides a method of producing viable population of cells, comprising: (a) freezing a composition comprising the population of cells, an aqueous component, about 0.5 M to about 2 M glycerol, about 0.2 M to about 0.6 M trehalose, and about 2 mg/mL to about 20 mg/mL polysaccharide, wherein the freezing occurs below about −30° C.; (b) removing the aqueous component from the frozen composition to produce a population of lyophilized cells, wherein the aqueous component in the population of lyophilized cells is less than about 5% w/w; and (c) resuspending the population of lyophilized cells in a reconstitution agent to form a reconstituted composition, wherein at least about 5% of the cells in the reconstituted composition are viable. In some embodiments, the composition of (a) further comprises a hydrogel. In some embodiments, the hydrogel is a hyaluronan gel, alginate gel, agarose, or collagen gel. In some embodiments, the population of cells in (a) is suspended in the hydrogel.

In some embodiments, the disclosure provides a method of producing a population of lyophilized cells, comprising: (a) freezing a composition comprising a population of cells and an aqueous component in a hydrogel matrix; and (b) removing at least about 90% of the aqueous component from the frozen composition to produce the population of lyophilized cells.

In some embodiments, the disclosure provides a method of producing a population of reconstituted viable cells, comprising: (a) freezing a composition comprising a population of cells and an aqueous component in a hydrogel matrix; (b) removing at least about 90% of the aqueous component from the frozen composition to produce the population of lyophilized cells, and (c) resuspending the population of lyophilized cells in a reconstitution agent to form a reconstituted composition, wherein at least about 1% of the cells are viable. In some embodiments, the hydrogel is a biocompatible hydrogel. In some embodiments, the hydrogel is a hyaluronan gel, alginate gel, agarose gel, or collagen gel. In some embodiments, the population of cells in (a) is suspended in the hydrogel. In some embodiments, the composition further comprises a polyol, a sugar, a polysaccharide, or combinations thereof. In some embodiments, the population of cells of (a) is about 1×10⁴ cells per mL to 1×10⁶ cells per mL. In some embodiments, the sugar is a disaccharide. In some embodiments, the disaccharide is selected from the group consisting of maltose, lactose, sucrose, lactulose, trehalose, cellobiose, isomaltose, melibiose, and gentiobiose. In some embodiments, the sugar is trehalose. In some embodiments, the polyol is selected from glycerol, propylene glycol and ethylene glycol. In some embodiments, the polyol is about 0.05 M to about 5 M. In some embodiments, the polyol is glycerol. In some embodiments, the glycerol is about 0.01 M to about 10 M in the composition. In some embodiments, the polysaccharide is about 1 mg/mL to about 25 mg/mL. In some embodiments, the polysaccharide is a polyglucan. In some embodiments, the polyglucan is a dextran. In some embodiments, the dextran has a molecular weight of about 10,000 Da to about 60,000 Da. In some embodiments, the composition of (a) is free of DMSO.

### BRIEF DESCRIPTION OF THE DRAWINGS

FIG. 1 outlines three possible steps to eliminate the solvent in a formulation by sublimation in the lyophilization process. Tg′ is the glass transition temperature. Teu is the eutectic melting temperature.

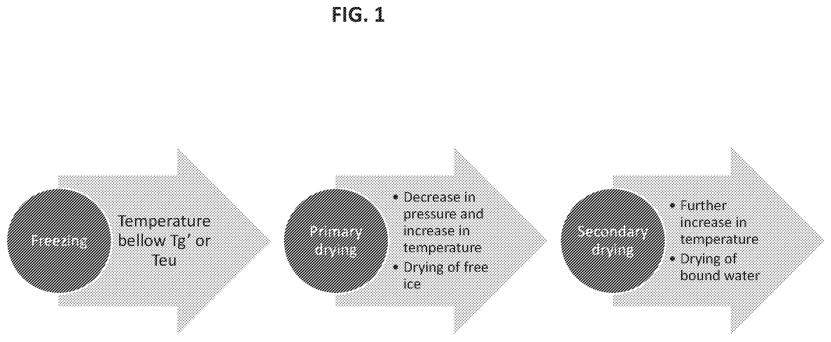

FIG. 2 demonstrates the outcome when (A) the product temperature during drying is below Tg′ (Visually impeccable lyophilization cake) and (B) the product temperature during drying is exceeds Tg′ (Collapsed lyophilization cake).

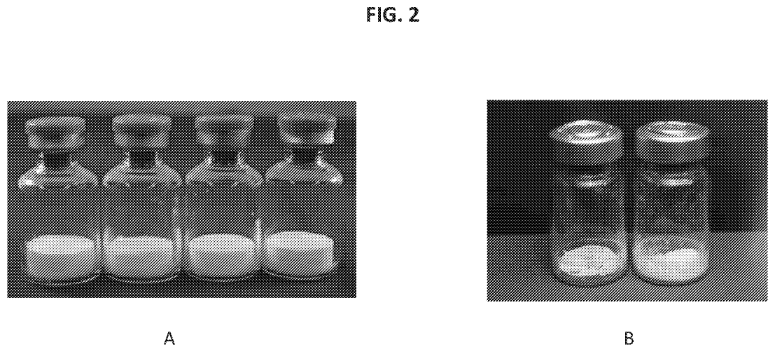

FIG. 3 provides an outline of the experiments described herein.

FIG. 4A represents a calibration curve for ALAMARBLUE® assay. Measurements were done in 6 replicates. FIG. 4B represents a growth curve of SK-N-AS cell line at different plating concentrations over 7 days. Measurements were done in 6 replicates.

FIG. 5: Cell viability after freeze thawing, measured with ALAMARBLUE® metabolic assay. Results are presented as percentage of calculated cell density, relative to control-untreated cells, representing 100%. Each condition was measured in 6 replicates.

FIG. 6: Cell viability after freeze thawing, measured with NucleoCounter, using DAPI and acridine orange staining. Each condition (formulation) was measured in 6 replicates.

FIG. 7A: Cell densities of plated cells compared to control—untreated cells, which did not undergo freeze thawing and were directly plated after harvesting from the flask. Each condition was measured in 6 replicates. FIG. 7B: Optimization of polyol propylene glycol concentration. Viability was measured with ALAMARBLUE®. Each condition was measured in 6 replicates.

FIG. 8A: Comparison of glycerol formulation in medium with current standard formulation for CTP cryopreservation—DMSO (5%), which has repeatedly delivered viabilities of above 80% (unpublished). DMSO (5%) was dissolved in medium. Each condition was measure in 5 replicates. FIG. 8B: Comparison of glycerol formulation in medium with current standard formulation for CTP cryopreservation—DMSO (5%), which has repeatedly delivered viabilities of above 80% (unpublished). DMSO (5%) was dissolved in medium. Each condition was measure in 5 replicates.

FIG. 9: Glycerol-trehalose formulations in hypertonic (FIG. 9A) and isotonic (FIG. 9B) conditions. Iso- and hypertonicity was achieved by varying the concentration of PBS in which the trehalose was dissolved. Conditions were measured in 6 replicates.

FIG. 10 represents data on the effect of tonicity of trehalose solutions. Glycerol with high trehalose concentration was worse than glycerol alone (FIG. 10A). When trehalose concentration was decreased so the conditions were close to isotonic, cell density increased (FIG. 10B). FIG. 10C: Hypertonic vs isotonic trehalose and DMSO solution were comparable. Both conditions were measured in 6 replicates.

FIG. 11: Negative impact of decreased concentration of PBS on cell post freeze-thaw viability in propylene glycol and ethylene glycol. All conditions were measured in 6 replicates.

FIG. 12: Freeze thawing with supplements from cell medium. Cell densities of plated cells after freeze-thawing in formulations with propylene glycol (PG) and medium supplements. Cells were incubated overnight before the measurement. Samples were in six replicates per condition.

FIG. 13A: Cell survival of thermal treatment for introduction of trehalose into cells. FIG. 13B: Survival of freeze-thaw process after thermal treatment. All the results are presented as percentage of cell densities of treated cells relative to freshly harvested cells.

FIG. 14 represents the results of collagen coating of glass vials with two protocols (A and B). Microscope pictures (5×magnification) of 2D attached cells on collagen coated vials after 1 day of incubation. FIG. 14A: Vial was coated with collagen according to protocol A. FIG. 14B: Vials were coated according to protocol B.

FIG. 15 Microscope pictures (5× magnification) of 3D cell cultures in hydrogels in Transwell® inserts.

FIG. 16 ALAMARBLUE® for cell viability and 3D cell growth in hydrogel scaffold of Hyaluronic acid (HA) and HYSTEM-HP® inserts with cells and a blank control—hydrogel scaffold without cells. Cells were incubated with ALAMARBLUE® reagent for 24 h. Fluorescence was measured after 4 h and 24 h of incubation. Samples were measured in duplicates.

FIG. 17 Microscope pictures (5× magnification) of 3D cell growth inside HYSTEM® and HYSTEM-C® hydrogel scaffolds at initial (FIG. 17A and FIG. 17C), and after 14 days (FIG. 17B and FIG. 17C), respectively.

FIG. 18A: Attached 2D cultured cells after FT in cryopreservative solution. FIG. 18B: Cell density of plated and attached cells after FT. Density was measured after one day of incubation. Cryopreservative solution (cryop.) includes 1M glycerol and 0.1M trehalose in PBS, concentration of dextran was of 10 mg/mL. Samples included minimum of 3 replicates.

FIG. 19: ALAMARBLUE® assay results of before and after FT measurements. Label “*” marks the post thaw results, that were found to be statistically different than the result of blank measurement. Two-tailed t-test assuming unequal variances was used to determine significance.

FIG. 20 shows an exemplary process for isolation and preparations of cells for allogenic cell therapy from Farid, S. S. & Jenkins, M. J. Chapter 44—Bioprocesses for Cell Therapies. in Biopharmaceutical Processing (eds. Jagschies, G., Lindskog, E., Ła̧cki, K. & Galliher, P.) 899-930 (Elsevier, 2018).

FIG. 21: Suspended cell formulations with cryopreservants after lyophilization process. 2R vials were used and fill volume was 1 mL. A: 1M glycerol and 0.1M trehalose, B: 1M propylene glycol and 0.1M trehalose, C: 0.5M glycerol and 0.1M trehalose, D: 1M glycerol, 0.1M trehalose and 10 mg/mL dextran.

FIG. 22 shows collapsed lyophilization cake of 2D attached cells on collagen coated vials with formulations containing (A) 1M glycerol+0.1M trehalose+10 mg/mL dextran, and (B) 1M glycerol+0.4 M Trehalose+10 mg/mL dextran.

FIG. 23 shows the cell survivability of the lyophilized formulations of FIG. 22.

FIG. 24 shows lyophilization of formulations with suspended population of cells in (A) hyaluronic acid (15 mg/mL), (B) 1M glycerol+0.1M trehalose+10 mg/mL dextran, (C) 1M glycerol+0.4 M trehalose, and (D) 1M glycerol+0.4M trehalose+10 mg/mL dextran.

FIG. 25 shows the cell survivability of the lyophilized formulations of FIG. 24.

FIG. 26 shows (A) hydrogels before lyophilization (B) hydrogel after lyophilization with I: HyStem-HP without cryoprotectants. II: HyStem-HP with 1M glycerol and 0.1M trehalose. III: HyStem-HP with 1M glycerol, 0.1M trehalose and 10 mg/mL of dextran.

FIG. 27 shows visually impeccable lyophilization cake of 3D cell culture formulations in hydrogels (A) HyStem and (B) HyStem-C without any cryoprotectants.

FIG. 28 shows the cell survivability of the lyophilized formulations of FIG. 27 in Hy Stem and HyStem-C without any cryoprotectants.

### DETAILED DESCRIPTION OF THE INVENTION

The present disclosure relates lyophilization of cells that remain viable after reconstitution.

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any method, system, host cells, temperatures, and/or composition of the present disclosure. Furthermore, compositions, systems, host cells, and/or temperatures of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As discussed in the Background section, the most conventional method for long-term storage of cells is cryopreservation, with its accompanying drawbacks. Many of these drawbacks can be eliminated if the cells can be dried and stored at ambient or refrigerated conditions like other biological drug products. The present disclosure provides methods for producing lyophilized cells that can be stored for a period of time, e.g., at refrigerated temperatures or above.

In some embodiments, the disclosure provides a method of producing a population of lyophilized cells, comprising: (a) freezing a composition comprising a population of cells, an aqueous component, a polyol, a sugar, and a polysaccharide; and (b) removing at least about 90% of the aqueous component from the frozen composition to produce the population of lyophilized cells.

In some embodiments, the disclosure provides a method of producing a population of reconstituted viable cells, comprising: (a) freezing a composition comprising a population of cells, an aqueous component, a polyol, a sugar, and a polysaccharide; (b) removing at least about 90% of the aqueous component from the frozen composition to produce the population of lyophilized cells, and (c) resuspending the population of lyophilized cells in a reconstitution agent to form a reconstituted composition, wherein at least about 1% of the cells are viable.

In some embodiments, the disclosure provides a composition comprising a population of cells, an aqueous component, a polyol, a sugar, and polysaccharide, wherein the composition is less than about 10% (vol/vol) water, and wherein the population of cells can be resuspended, wherein at least about 1% the cells in the population of cells are viable when resuspended.

In some embodiments, the disclosure provides a composition comprising a population of cells, an aqueous component, glycerol, trehalose, and polysaccharide, wherein the composition is less than about 10% (vol/vol) water, and wherein the population of cells can be resuspended, wherein at least about 1% the cells in the population of cells are viable when resuspended.

In some embodiments, the disclosure provides a composition comprising a population of cells, an aqueous component, about 0.5 M to about 2 M polyol, about 0.2 M to about 0.6 M sugar, and about 2 mg/mL to about 20 mg/mL polysaccharide, wherein the population of cells is about 2×10⁵ cell/cm² to about 3×10⁵ cells/cm².

In some embodiments, the disclosure provides a composition comprising a population of cells, an aqueous component, about 0.5 M to about 2 M glycerol, about 0.2 M to about 0.6 M trehalose, and about 2 mg/mL to about 20 mg/mL polysaccharide, wherein the population of cells are about 2×10⁵ cell/cm² to about 3×10⁵ cells/cm².

In some embodiments, the disclosure provides a method of lyophilizing a population of cells, comprising: (a) freezing a composition comprising the population of cells, an aqueous component, about 0.5 M to about 2 M glycerol, about 0.2 M to about 0.6 M trehalose, and about 2 mg/mL to about 20 mg/mL polysaccharide, wherein the freezing occurs below about −30° C.; and (b) removing the aqueous component from the frozen composition to produce a population of lyophilized cells, wherein the aqueous component in the population of lyophilized cells is less than about 5% w/w.

In some embodiments, the disclosure provides a method of producing viable population of cells, comprising: (a) freezing a composition comprising the population of cells, an aqueous component, about 0.5 M to about 2 M polyol, about 0.2 M to about 0.6 M sugar, and about 2 mg/mL to about 20 mg/mL polysaccharide, wherein the freezing occurs below about −30° C.; (b) removing the aqueous component from the frozen composition to produce a population of lyophilized cells, wherein the aqueous component in the population of lyophilized cells is less than about 5% w/w; and (c) resuspending the population of lyophilized cells in a reconstitution agent to form a reconstituted composition, wherein at least about 1% of the cells in the reconstituted composition are viable.

In some embodiments, the disclosure provides a method of producing viable population of cells, comprising: (a) freezing a composition comprising the population of cells, an aqueous component, about 0.5 M to about 2 M glycerol, about 0.2 M to about 0.6 M trehalose, and about 2 mg/mL to about 20 mg/mL polysaccharide, wherein the freezing occurs below about −30° C.; (b) removing the aqueous component from the frozen composition to produce a population of lyophilized cells, wherein the aqueous component in the population of lyophilized cells is less than about 5% w/w; and (c) resuspending the population of lyophilized cells in a reconstitution agent to form a reconstituted composition, wherein at least about 1% of the cells in the reconstituted composition are viable.

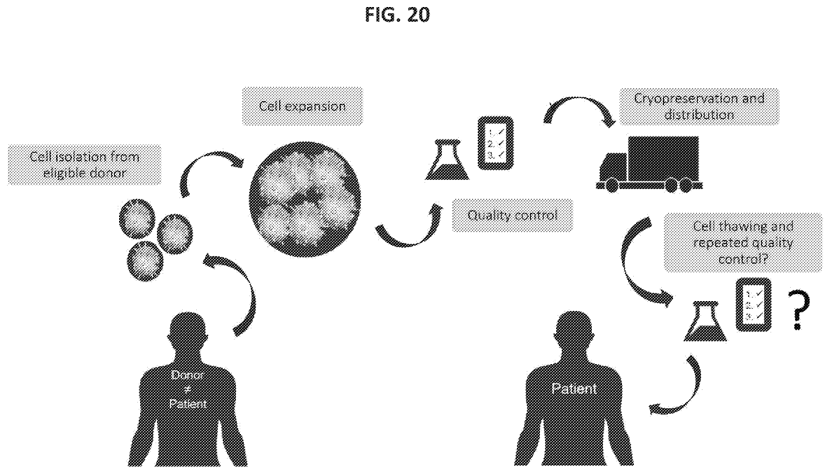

In some embodiments, the present disclosure provides methods of producing lyophilized cells for cell therapy. An exemplary process for preparation of cells for cell therapy includes the process for allogenic cell therapy is illustrated in FIG. 20. Allogenic cell therapy includes cell isolation from an eligible donor (e.g., a non-patient donor); cell expansion; quality control; cryopreservation and distribution; cell thawing and repeated quality control; and then introducing the cells to the patient. In some embodiments of the present disclosure, the present disclosure is directed to a process wherein the cells are lyophilized. In some embodiments, the present disclosure is directed to a process wherein the cells can be distributed and/or stored at room temperature. In some embodiments, the present disclosure is directed to a process wherein the cells can be reconstituted and become viable. In some embodiments, the present disclosure is directed to a process wherein the cells are reconstituted and the need to conduct quality control assays on the cells is minimized or eliminated before introducing the cells to a patient. In some embodiments, the disclosure is directed to a method of isolating a cell from an eligible donor (e.g., a non-patient donor); performing cell expansion; lyophilizing the cells, reconstituting the cells, and then introducing the cells to a patient.

### Lyophilization Overview

“Lyophilization” (also termed “freeze-drying” and “cryodesiccation”) and variants thereof broadly refers to freezing a substance and then reducing the concentration of one of the solutes, typically water, by sublimation and desorption (i.e., “drying”). In some embodiments, the freezing is the transition of the substance, e.g., a composition comprising one or more components, from liquid state to solid state. In some embodiments, the drying comprises applying vacuum to the frozen substance, e.g., frozen composition. In some embodiments, the drying comprises lowering pressure, applying a vacuum, or both, to the frozen composition.

Lyophilization can be used for drying of thermally labile compounds, such as microorganisms and proteins. Instead of using evaporation mechanism of solvent elimination, which is common with small molecules, it employs sublimation. It mainly consists of three process steps: freezing, primary drying and secondary drying. Lyophilization is accomplished by placing the composition to be lyophilized in a lyophilization chamber, where the freezing, drying and ultimately stoppering under inert atmosphere takes place. During drying, water vapor passes through a connecting duct into a condenser. The temperature in the condenser is lower than the temperature of the lyophilization chamber such that the water vapor turns into ice.

### Freezing

Freezing can have a large impact on the rate and homogeneity of drying. Freezing of the solvent can be subdivided into cooling, phase change and solidification. Formulation does not freeze at the equilibrium freezing temperature, but at a lower one. This is either because no ice crystal nuclei are yet formed in the solution, or because there are appreciable temperature differences within the system. The degree at which the phase change occurs, starting with primary and then secondary nucleation, is called the degree of supercooling. Primary nucleation is defined as the appearance of the first ice nucleus, while secondary nucleation is characterized by formation of additional nucleation sites. After phase change stage, the solidification starts. Here the ice crystals start growing and the amount of liquid water phase is reduced, leading to increasing solute concentration. When the critical concentration is exceeded, the concentrated solution undergoes either eutectic freezing or vitrification. The temperature at which this occurs is called the eutectic temperature (Tₑᵤ) for crystalline systems and the glass transition temperature of the maximally freeze-concentrated solution (T_{g}′) for amorphous systems. Below these temperatures the system is considered completely solidified. Different crystal sizes and morphologies form depending on the degree of supercooling. This, upon sublimation during drying step, affects the pore sizes in the dry products. Small ice crystals form smaller pores, which causes higher resistance to mass transfer and therefore slower primary drying. However, because of larger surface area, the secondary drying in this case, proceeds with a higher rate. Vice versa is true for larger ice crystals. The conclusion is, that faster crystallization, which leads to smaller crystals, causes slower primary drying but faster secondary drying.

### Primary Drying

After the formulation is in its frozen state, the chamber is evacuated to a pressure close to vacuum and the temperature of the shelf is slowly increased to a temperature close, but below, collapse temperature (T_{c}). T_{c} is usually several degrees higher than T_{g}′ and is a temperature, where the cake loses its structure. The correlation between the collapsed cake and product stability is still debatable. However, visual collapse may cause the drug product to be rejected after visual inspection by a manufacturer.

The driving force of primary drying is the gradient between ice vapor pressure and chamber pressure. The vapor pressure of ice is dependent on the temperature of the product, which is the most important parameter in the process. The product temperature is controlled by both changing the shelf temperature and chamber pressure. The higher shelf temperature leads to higher product temperature and hence to faster sublimation. On the other hand, since sublimation is an endothermic process, a lower chamber pressure leads to cooling of the product, which causes a lower rate of sublimation.

When drying proteins, it may be important to use protectants against water loss and low temperatures in addition to bulking agents. Common substances used as protectants include sorbitol, sucrose, trehalose, mannitol, polyvinylpyrrolidone (PVP), dextrose and glycine. Most of these compounds have an additional positive effect of increasing T_{g}′ of the formulation. This is desirable, because the process can be run at higher temperatures, which makes it faster and therefore more efficient. Bulking agents provide a nice appearance and structure of the product and some also serve as protectants—mannitol is an example of this. Crystalline bulking agents are desirable, because they tend to provide elegant cake structures.

### Secondary Drying

Free ice is sublimated during primary drying, which is the longest step in the lyophilization process. However, some of the water remains bound to the product and therefore needs to be removed with a more aggressive treatment. This is achieved during secondary drying, where the chamber pressure remains the same as in primary drying and shelf temperature is increased typically to 30-50° C. At this point, it is possible to raise the temperature to such levels, because there is no more free ice in the vial and so there is no danger of collapse of the lyophilization cake.

In some embodiments, the freezing of the composition comprising cells, an aqueous component, and a lyophilization agent comprises placing containers containing the composition in liquid nitrogen. In some embodiments, the freezing of the composition comprises placing containers containing the composition in a −80° C. freezer. In some embodiments, the freezing of the composition comprises placing containers comprising the composition in a freezing container, e.g., the MR. FROSTY Freezing Container from THERMO SCIENTIFIC. In some embodiments, the freezing of the composition comprises placing containers comprising the composition on a pre-cooled shelf in a freezer. In some embodiments, the containers comprise a cryogenic vial or an injection glass vial, e.g., a 2R glass vial.

In some embodiments, the freezing of the composition does not use liquid nitrogen. In some embodiments, the freezing occurs at between about −30° C. to about −100° C. In some embodiments, the freezing occurs at between about −60° C. to about −90° C. In some embodiments, the freezing occurs at between about −70° C. to about −80° C. In some embodiments, the freezing occurs at about −60° C. , about −65° C., about −70° C., about −75° C., about −80° C., about −85° C., or about −90° C. In some embodiments, the freezing occurs at between about −10° C. to about −100° C. In some embodiments, the freezing occurs at between about −20° C. to about −90° C. In some embodiments, the freezing occurs at between about −40° C. to about −60° C. In some embodiments, the freezing of (a) lowers the temperature of the composition to about −50° C. In some embodiments, the freezing of (a) lowers the temperature of the composition to about −50° C., the aqueous component is removed at a pressure of a chamber pressure of about 20 mTorr to about 40 mTorr.

In some embodiments, the rate at which the temperature of the composition comprising a population of cells is lowered can affect the viability of the cells. The rate of cooling will be dependent on numerous factors, including the cooling apparatus, the container, the composition, and the volume of the composition. In some embodiments, the temperature of the composition is reduced about 1° C. to about 50° C. per second, about 2° C. to about 40° C. per second, about 3° C. to about 30° C. per second, about 4° C. to about 20° C. per second, or about 5° C. to about 10° C. per second. In some embodiments, the temperature of the composition is reduced about 1° C. to about 50° C. per minute, about 2° C. to about 40° C. per minute, about 3° C. to about 30° C. per minute, about 4° C. to about 20° C. per minute, or about 5° C. to about 10° C. per minute. In some embodiments, the temperature of the composition comprising a population of cells does not exceed the T_{g} of the composition during the freezing process.

In some embodiments, the removing the aqueous component in the method comprises lowering pressure, applying heat, or both, to the frozen composition to remove the aqueous component. In some embodiments, the lowering pressure comprises applying vacuum to the frozen composition. In some embodiments, the lowering pressure comprises lowering the pressure to 10 torr, e.g. less than about 8 torr, less than about 5 torr, less than about 2 torr, or less than about 1 torr. In some embodiments, the lowering pressure comprises lowering the pressure to less than about 0.8 torr. In some embodiments, the lowering pressure comprises lowering the pressure to less than about 0.4 torr.

In some embodiments, the removing the aqueous component comprises a primary drying step and a secondary drying step. In some embodiments, the primary drying step comprises lowering pressure to remove the aqueous component. In some embodiments, the secondary drying step comprises applying heat to remove the aqueous component. In some embodiments, the primary drying step comprises sublimation (i.e., transition from solid phase to gas phase) of the aqueous component of the composition. In some embodiments, the secondary drying step comprises desorption of the aqueous component of the composition. When used in the context of lyophilization, “desorption” refers to the disruption of physio-chemical interactions between the aqueous component (e.g., water molecules) and one or more components of the frozen composition. In some embodiments, the pressure and temperature are selected such that the aqueous component of the composition is capable of sublimation and/or desorption.

In some embodiments, the applying heat in the secondary drying step comprises heating the composition to greater than −20° C. In some embodiments, the applying heat comprises heating the composition to greater than −10° C. In some embodiments, the applying heat comprises heating the composition to greater than 0° C.

In some embodiments, the lyophilization removes the solute, e.g., water or aqueous component, from the composition. One of skill in the art will appreciate that “lyophilized cells” may still comprise some amount of water. In some embodiments, the amount of solute remaining in the population of lyophilized cells can be a factor in the cells maintaining their viability when the cells are reconstituted. In some embodiments, the population of lyophilized cell comprises less than 10% w/w of aqueous component. In some embodiments, the population of lyophilized cell comprises less than 5% w/w of aqueous component. In some embodiments, the population of lyophilized cell comprises less than 10%, less than 9%, less than 8%, less than 7%, less than 6%, less than 5%, less than 4%, less than 3%, less than 2%, or less than 1% w/w of aqueous component. In some embodiments, the composition comprising a lyophilized mixture of a cell and a lyophilization agent selected from glycerol, propylene glycol, or combinations thereof comprise less than 10% of aqueous component. In some embodiments, the composition comprising a lyophilized mixture of a cell and a lyophilization agent selected from glycerol, propylene glycol, or combinations thereof comprise less than 5% of aqueous component. In some embodiments, the composition comprising a lyophilized mixture of a cell and a lyophilization agent selected from glycerol, propylene glycol, or combinations thereof comprise less than 10%, less than 9%, less than 8%, less than 7%, less than 6%, less than 5%, less than 4%, less than 3%, less than 2%, or less than 1% w/w of aqueous component.

In some embodiments, the lyophilization is performed in a device capable of both freezing the composition and removing the aqueous component, e.g., a freeze-dryer such as the FREEZONE freeze dryer from LABCONCO, the FREEZEMOBILE, VIRTIS, and HULL freeze dryers from SP SCIENTIFIC, and the STELLAR, REVO, MAGNUM, and EPIC freeze dryers from MILLROCK TECHNOLOGY. In some embodiments, the freezing the composition and the removing the aqueous component are performed in separate devices, e.g., a freezer, e.g., −80° C. freezer, or liquid nitrogen for freezing the composition, and a vacuum system for removing the aqueous component.

In some embodiments, the substance comprises a biological product, e.g., one or more cells. In some embodiments, the substance comprises a composition comprising cells, an aqueous component, and a lyophilization agent.

In some embodiments, the aqueous component that is removed by lyophilization comprises buffer. In some embodiments, the buffer comprises phosphate buffer, Tris buffer, acetate buffer, bicarbonate buffer, histidine buffer, citrate buffer or combinations thereof. In some embodiment, the aqueous component comprises cell culture medium, i.e., the population of cells that are frozen are in a cell culture medium. In some embodiments, the cell culture medium is free of serum.

In some embodiments, the removing the aqueous component comprises lowering pressure, applying heat, or both, to the frozen composition to remove the aqueous component. In some embodiments, the removing the aqueous component comprises a primary drying step and a secondary drying step. In some embodiments, the removing the aqueous component comprises greater than two drying steps. In some embodiments, the primary drying step comprises lowering pressure to remove the aqueous component. In some embodiments, the secondary drying step comprises applying heat to remove the aqueous component.

In some embodiments, the skilled artisan can alter the tonicity of the solution prior to freezing the population of cells. In some embodiments, the composition is an isotonic solution. In some embodiments, the composition is a hypertonic solution.

In some embodiments, the cells are lyophilized on a 2-dimensional surface, e.g., on the surface of a plate or a container. In some embodiments, the lyophilization is performed on a surface, e.g., in a container, with or without collagen coating. In some embodiments, the surface, e.g., the container, is a glass or plastic surface/container without or without membrane for cell attachment or growth.

In some embodiments, the cells are lyophilized on a 3-dimensional matrix. In some embodiments, the lyophilization is performed on a 3D matrix in a container, e.g., with or without collagen coating.

In some embodiments, the composition comprising cells, an aqueous component, and a lyophilization agent further comprises a matrix. Non-limiting examples of matrices include, e.g., collagen (such as, e.g., Type I, Type II, or Type IV collagen), elastin, fibronectin, laminin, vitronectin, cadherin, tenascin-C, and other matrix-derived peptides. In some embodiments, the population so cells are suspended in a matrix. In some embodiments, the matrix is a hydrogel. Thus, in some embodiments, the population cells are suspended in a hydrogel. A hydrogel is a network of polymer chains that are hydrophilic, sometimes found as a colloidal gel in which water is the dispersion medium. In some embodiments, the hydrogel is a biocompatible hydrogel. In some embodiments, the hydrogel is a hyaluronan gel, alginate gel, agarose gel, collagen gel, or combination thereof. In some embodiments, the hydrogel is a combination of hyaluronan gel, alginate gel, agarose gel, or collagen gel. In some embodiments, the population of cells are suspended in the hydrogel. In some embodiments, the freezing of the population of cells is performed either in suspension or attached in a container. In some embodiments, the freezing of the population of cells is performed with the population of cells suspended in a hydrogel.

In some embodiments, the hydrogel is a HYSTEM™ Gel, available from Sigma Aldrich. HYSTEM™ can include HYSTEM™ which is composed of thiol modified HA (GLYCOSIL®) and thiol reactive crosslinker (EXTRALINK®). See, e.g., Chen D, et al., Eye (Load), 2017 June; 31(6):962-971; Devarasetty M,, et al., Biofabrication, 2017 Jun. 7; 9(2):021002; Mannino R G, et al., Lab Chip, 2017 Jan. 31; 17(3):407-414; Chen X, et al., J Tissue Eng Regen Med, 2016 May; 10(5):437-46; and Engel B J,, et al., Adv Healthc Mater, 2015 Aug. 5; 4(11):1664-74. The crosslinker allows for the gelification process (without it, HA solution will stay liquid). HYSTEM™ can also include HYSTEM™ C includes GLYCOSIL® and EXTRALINK® but also thiol modified denatured collagen fibris called GELIN-S® to accommodate some cell attachment needs (e.g. Stem cells). HYSTEM™ can also include HYSTEM™ HP (HYSHP020-1KT, sigmaaldrich.com/catalog/product/sigma/hyshp020?lang=en&region=US) which includes all of HYSTEM™ C plus heparin sulfate to ensure growth factor release at the immediate proximity of cells. In some embodiments, the hydrogel comprises more than one HYSTEM™ gel. In some embodiments, the hydrogel comprises a HYSTEMTM gel that has been further processed, e.g., has been chemically modified. In some embodiments, the hydrogel comprises a HYSTEMgel and an alginate gel, agarose gel, collagen gel and/or a different hyaluronan gel.

In some embodiments, the component is collagen. In some embodiments, the composition comprises a synthetic polymer that facilitates cell attachment. In some embodiments, the synthetic polymer comprises poly-lysine, poly-L-ornithine, or combinations thereof.

The present disclosure provides for population of lyophilized cells. As used herein, the term population refers to one or more cells, e.g., cells isolated from an individual or cells propagated in cell culture. As used herein, the terms “cell” or “cells” refer to one or more cells, e.g., a population of cells. In some embodiments, the cell is a eukaryotic cell. In some embodiments, the cell is a bacterial cell or archaeal cell. In some embodiments, the cell is a mammalian cell or a plant cell. In some embodiments, the cell is a human cell. In some embodiments, the cell is from a domesticated animal, e.g., a cow, horse, dog, or cat. In some embodiments, the cell is a stem cell. In some embodiments, the cell is a pluripotent stem cell, an embryonic stem cell, a mesenchymal stem cell, or a hematopoietic stem cell. In some embodiments, the cell is a mesenchymal stem cell (MSC). In some embodiments, the cell is an induced pluripotent stem cell (iPSC). In some embodiments, the cell is a germ cell such as, e.g., a T-cell, a fibroblast, a chondrocyte, a hepatocyte, an enterocyte, an erythrocyte, a neural cell, an epithelial cell, or an endothelial cell. In some embodiments, the cell is an immortalized cell. In some embodiments, the cell is a neural cell, e.g., in some embodiments, the cell is a neuroblastoma cell. In some embodiments, the neuroblastoma cells are SK-N-AS cells.

In some embodiments, the cell is a bacterial cell. Examples of such bacterial cells include, but are not limited to, E. coli, S. aureus, V. cholerae, S. pneumoniae, B. subtilis, C. crescentus, M genitalium, A. fischeri, Synechocystis, P. fluorescens, A. vinelandii, S. coelicolor. In some embodiments, the bacterial cell is of bacteria used in preparation of food and/or beverages. Non-limiting exemplary genera of such cells include, but are not limited to, Acetobacter, Arthrobacter, Bacillus, Bifidobacterium, Brachybacterium, Brevibacterium, Carnobacterium, Corynebacterium, Enterococcus, Gluconacetobacter, Hafnia, Halomonas, Kocuria, Lactobacillus (including L. acetotolerans, L. acidipiscis, L. acidophilus, L. alimentarius, L. brevis, L. bucheri, L. casei, L. curvatus, L. fermentum, L. hilgardii, L. jensenii, L. kimchii, L. lactis, L. paracasei, L. plantarum, and L. sakei), Leuconostoc, Microbacterium, Pediococcus, Propionibacterium, Weissella, and Zymomonas.

In some embodiments, the cell is a eukaryotic cell. In some embodiments, the eukaryotic cell is an animal or human cell. In some embodiments, the eukaryotic cell is a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. In some embodiments, the eukaryotic cells are CHO-cell lines. In some embodiments, the eukaryotic cell is a CHO cell. In some embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. Eukaryotic cells can also be avian cells, cell lines or cell strains, such as, for example, EBX cells, EB14, EB24, EB26, EB66, or Ebvl3.

In some embodiments, the eukaryotic cell is a human cell. In some embodiments, the human cell is a stem cell. In some embodiments, the methods provided herein are advantageous for producing lyophilized stem cells that are viable upon reconstitution, e.g., for use in cell therapy. The stem cells can be, for example, pluripotent stem cells, including embryonic stem cells (ESCs), adult stem cells, induced pluripotent stem cells (iPSCs), tissue specific stem cells (e.g., hematopoietic stem cells, neural stem cells, epithelial stem cells, skin stem cells, and the like) and mesenchymal stem cells (MSCs). In some embodiments, the stem cell is a mesenchymal stem cell (MSC). In some embodiments, the MSC is obtained from the bone marrow, cord blood, peripheral blood, fallopian tube, liver, and/or lung of a human subject. In some embodiments, the stem cell is an induced pluripotent stem cell (iPSC). In some embodiments, the iPSC has at least one vector capable of expressing one or more pluripotency factors. In some embodiments, the iPSC is derived from a fibroblast, keratinocyte, peripheral blood mononuclear cell (PBMCs), hepatocytes, neuronal cell, B cell, muscle cell, adrenal cell, and/or renal epithelial cell of a human subject, or any other type of cell known to be suitable for becoming an induced pluripotent stem cell. In some embodiments, the human cell is a differentiated form of any of the cells described herein. In some embodiments, the cell comprises at least one vector capable of expressing one or more pluripotency factors, but the pluripotency factors have not yet been expressed before lyophilization, thus the cell has not been induced to pluripotency yet. In some embodiments, the eukaryotic cell is a cell derived from any primary cell in culture.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). In embodiments, the cell is cell from the eye. In some embodiments, the cell is a retinal cell, a scleral cell, a choroidal epithelial cell, a macrophage cell, or an immune cell.

In some embodiments, the cell is obtained from a cell line. Non-limiting examples of cell lines include MOLT-4 (differentiated or undifferentiated), Jurkat, HL60 (differentiated or undifferentiated), U-937 (differentiated or undifferentiated), HDLM-2, THP-1 (differentiated or undifferentiated), GA10, Ramos, HUVEC, PANC-1, Expi293, HaCat, HCT-15, H-2228, peripheral blood mononuclear cells (PBMCs), KU-812, MC-04, HT-1376, TT, HCT-1116, MCF-7, Calu-3, and the like. Exemplary monocytic cell lines include THP-1, differentiated THP-1, HL60, and differentiated HL60. An exemplary NK cell line is NK92. Exemplary T cell lines include Jurkat and Molt-4. An exemplary B cell line is GA-10. Exemplary endothelial cell lines include HUVEC and differentiated HUVEC. Exemplary hepatocytic cell lines include HepG2 and differentiated HepG2. Exemplary epithelial cell lines include A549, A431, Caco-2, HT29, LNCap, SKOV3, SW480, PC3, MDMB-468, MDMB-231, MCF7, HT-1376, PANC-1, HCT15, Calu-3, Skov3, Bewo, K562, and HeLa. Further additional cell lines include, e.g., HT-29 sARPE-19, SH-SY5Y, and U87-MG.

Further examples of cells include a T cell, a B cell, a dendritic cell, an NK cell, a monocyte, a macrophage, a granulocyte, a platelet, an erythrocyte, an endothelial cell (e.g., an aortic endothelial cell), an epithelial cell, a stem cell precursor cell, a mesenchymal stem cell, a hematopoietic stem cell, a leukocyte, a senescent cell, an adipose cell, a hepatocyte, a myocyte, or a skeletal muscle cell. T cells include, e.g., helper T cells, such as the subtypes Th1, Th2, Th9, Th17, Th22, and Tfh; regulatory T cells; killer T cells; γδ TCR+ T cells; and natural killer T cells. Adipose cells include, e.g., normal adipocytes, diabetic adipocytes, omental adipocytes, MSC-derived adipocytes, preadipocytes, and omental preadipocytes.

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, e.g., potatoes; plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In some embodiments, the cells include a cell that is in a microbiota. In some embodiments, the cells are a combination of cells that comprise a microbiota. In some embodiments, the cells comprise a full array of microorganisms in the microbiota that live on and/or in an organism, e.g., humans, a domesticated animal (e.g., a cow, pig, chicken, horse, etc.), or a zoo animal. In some embodiments, the microbiota includes bacteria, archaea (primitive single-celled organisms), fungi, and even some protozoans and nonliving viruses. In some embodiments, the microbiota is a gastrointestinal microbiota (e.g., a esophageal, stomach, and/or intestinal microbiota), an oral microbiota, a urinary tract microbiota, a nasal microbiota, respiratory microbiota, a skin microbiota, a vaginal microbiota, a rectal micobiota, or a combination thereof. In some embodiments, the microbiota is an infant's microbiota. In some embodiments, the microbiota is an adult's microbiota.

In some embodiments, the disclosure provides for a method of producing a population of lyophilized cells comprising a partial array or a full array of a microbiota, comprising: (a) freezing a composition comprising a population of cells form a microbiota, an aqueous component, a polyol, a sugar, and a polysaccharide; and (b) removing at least 90% of the aqueous component from the frozen composition to produce the population of lyophilized cells. In some embodiments, the disclosure provides a method of producing a population of reconstituted viable cells comprising a partial array or a full array of a microbiota, comprising: (a) freezing a composition comprising a population of cells from a partial array or a full array of a microbiota, an aqueous component, a polyol, a sugar, and a polysaccharide; (b) removing at least 90% of the aqueous component from the frozen composition to produce the population of lyophilized cells, and (c) resuspending the population of lyophilized cells in a reconstitution agent to form a reconstituted composition comprising a partial array or a full array of a microbiota, wherein at least 1% of the cells are viable.

In some embodiments, the disclosure provides a method of producing a population of lyophilized cells comprising a partial array or a full array of a microbiota, comprising: (a) freezing a composition comprising a population of cells from a partial array or a full array of a microbiota and an aqueous component in a hydrogel matrix; and (b) removing at least about 90% of the aqueous component from the frozen composition to produce the population of lyophilized cells.

In some embodiments, the disclosure provides a method of introducing a microbiota to an organism, e.g., a human, by introducing cells reconstituted from the lyophilized cells as described herein. For example, in some embodiments, the lyophilized cells as described herein can form a solid form, e.g., a tablet, and the tablet can be introduced to gastrointestinal microbiota (e.g., a esophageal, stomach, and/or intestinal microbiota), an oral microbiota, a urinary tract microbiota, a nasal microbiota, respiratory microbiota, a skin microbiota, a vaginal microbiota, a rectal microbiota, or a combination thereof by ingestion or insertion. In some embodiments, the lyophilized cells as described herein can be reconstituted prior to being introduced to the organism. For example, the lyophilized cells comprising the microbiota can be reconstituted, and then introduced to gastrointestinal microbiota (e.g., an esophageal, stomach, and/or intestinal microbiota), an oral microbiota, a urinary tract microbiota, a nasal microbiota, respiratory microbiota, a skin microbiota, a vaginal microbiota, a rectal microbiota, or a combination thereof by ingestion or insertion. In a specific example, the lyophilized cells comprising the microbiota can be reconstituted, and then placed in an appropriate administrative form, e.g., a cream or liquid comprising the reconstituted microbiota could be applied to the skin, or a liquid, semi-solid, or solid comprising the reconstituted microbiota could be applied to the gastrointestinal tract.

The population of cells that are frozen can be at various concentrations. In some embodiments, the population of cells that are frozen are greater than 1×10² cells per mL, are greater than 5×10² cells per mL, are greater than 1×10³ cells per mL, are greater than 5×10³ cells per mL, are greater than 1×10⁴ cells per mL, are greater than 5×10⁴ cells per mL, are greater than 1×10⁵ cells per mL, or are greater than 5×10⁵ cells per mL. In some embodiments, the population of cells that are frozen are less than 1×10⁷ cells per mL, less than 5×10⁶ cells per mL, or less than 1×10⁶ cells per mL. In some embodiments, the population of cells that are frozen is about 1×10⁴ cells per mL to 1×10⁶ cells per mL. In some embodiments, the population of cells that are frozen is about 1×10⁵ cells per mL to 4×10⁵ cells per mL. In some embodiments, the population of cells that are frozen is about 2×10⁵ cells per mL to 2.5×10⁵ cells per mL.

The present disclosure provides for lyophilization agents that assist in stabilizing the cells such that they remain viable during and after lyophilization. Lyophilization agent refers to a substance that may aid in the lyophilization process, e.g., by stabilizing one or more components of the composition from degradation or undesired precipitation, promoting removal of the aqueous component, and/or protecting the cells from lysis or damage during the freezing and/or removing the aqueous component. In some embodiments, the lyophilization agent of the composition comprises a polyol, a sugar, and a polysaccharide

Polyol, as described herein, can include polyalcohols having two or more hydroxy groups linked to a carbon chain having from two to twelve carbon atoms. In some embodiments, the term polyol includes polyalcohols having two or more hydroxy groups linked to a carbon chain having from two to eight carbons three to eight carbon. In some embodiments, the term polyol includes polyalcohols having two or more hydroxy groups linked to a carbon chain having from two to six carbons, such as, for example, glycerol, propylene glycol, eherythritol, arabitol, xylitol, ribitol, mannitol, sorbitol, galactitol, and inositol. In some embodiments, the term polyol includes polyalcohols having two or more hydroxy groups linked to a straight (e.g., non-ring forming) carbon chain having from two to six carbons. In some embodiments the polyol is glycerol, propylene glycol, or ethylene glycol. In some embodiments, the polyol is glycerol. In some embodiments, the polyol is polyethylene glycol.

The polyol can be present in various concentrations in the composition comprising the population of cells. In some embodiments, the polyol is about 0.05 M to about 5 M in the composition comprising the population of cells. In some embodiments, the polyol is about 0.1 M to about 3 M. In some embodiments, the polyol is about 0.5 M to about 2 M. In some embodiments, the polyol is about 1 M to about 1.5 M. In some embodiments, the glycerol is about 0.01 M to about 10 M in the composition. In some embodiments, the glycerol is about 0.05 M to about 5 M. In some embodiments, the glycerol is about 0.1 M to about 3 M. In some embodiments, the glycerol is about 0.5 M to about 2 M. In some embodiments, the glycerol is about 1 M to about 1.5 M. In some embodiments, the glycerol is about 1.0 M, about 1.1 M, about 1.2 M, about 1.3 M, about 1.4 M, about 1.5 M, about 1.6 M, about 1.7 M, about 1.8 M, about 1.9 M, or about 2.0 M in the composition. In some embodiments, the glycerol is about 1.2 M to about 1.3 M in the composition. In some embodiments, the lyophilization reagent comprises glycerol, and the glycerol is about 1.23 M in the composition.

In some embodiments, the polyol comprises propylene glycol, and the propylene glycol is about 0.5% v/v to about 20% v/v of the composition. In some embodiments, the propylene glycol is about 1% v/v to about 10% v/v of the composition. In some embodiments, the propylene glycol is about 2% v/v to about 8% v/v of the composition. In some embodiments, the propylene glycol is about 3% v/v to about 6% v/v of the composition. In some embodiments, the propylene glycol is about 1% v/v, about 2% v/v, about 3% v/v, about 4% v/v, about 5% v/v, about 6% v/v, about 7% v/v, about 8% v/v, about 9% v/v, or about 10% v/v of the composition. In some embodiments, the propylene glycol is about 5% v/v of the composition.

Sugar, as described herein, refers to a monosaccharide, disaccharide, or trisaccharide, and can include both the D and the L forms. In some embodiments, the term sugar refers to a monosaccharide, e.g., glucose, fructose, galactose, mannose, ribose and deoxyribose. In some embodiments, the saccharides can include non-naturally occurring or semi-artificial monosaccharides. In some embodiments, the saccharides can include (i) Hexoses (contain 6 carbons) including: D- and L-allose, D- and L-altrose, D- and L-fucose, D- and L-gulose, D-sorbose, D-tagatose, (ii) Pentoses (contain 5 carbons): D- and L-arabinose, D- and L-lyxose, Rhamnose, D-ribose, Ribulose and its synthetic form sucroribulose, and D-xylose or wood sugar. In some embodiments, the term sugar refers to a disaccharide, e.g., maltose, lactose, sucrose, lactulose, trehalose, cellobiose, isomaltose, melibiose, and gentiobiose. The disaccharides can include various potential linkages. For example, in some embodiments, the disaccharide has a C₁-C₁ glycosidic linkage, e.g., an α(1→1)α linkage. However, in some embodiments, the disaccharide can include, e.g., an α(1→2)β, β(1→4) or α(1→4) linkage. In some embodiments, the sugar is trehalose.

The sugar can be present in various concentrations in the composition comprising the population of cells. In some embodiments, the sugar is about 0.05 M to about 5 M in the composition comprising the population of cells. In some embodiments, the sugar is about 0.1 M to about 3 M. In some embodiments, the sugar is about 0.1 M to about 1 M. In some embodiments, the sugar is about 0.2 M to about 0.6 M. In some embodiments, the sugar is about 0.4 M in the composition.

In some embodiments, the sugar is in the poration solution. In some embodiments, the sugar is about 0.05 M to about 5 M in the poration solution. In some embodiments, the sugar is about 0.1 M to about 3 M in the poration solution. In some embodiments, the sugar is about 0.1 M to about 1 M in the poration solution. In some embodiments, the sugar is about 0.2 M to about 0.6 M in the poration solution. In some embodiments, the sugar is about 0.4 M in the poration solution.

In some embodiments, the sugar is a disaccharide. In some embodiments, the disaccharide is about 0.05 M to about 5 M in the composition comprising the population of cells. In some embodiments, the disaccharide is about 0.1 M to about 3 M in the composition comprising the population of cells. In some embodiments, the disaccharide is about 0.1 M to about 1 M in the composition comprising the population of cells. In some embodiments, the disaccharide is about 0.2 M to about 0.6 M in the composition comprising the population of cells. In some embodiments, the disaccharide is about 0.4 M in the composition comprising the population of cells.

In some embodiments, the disaccharide is in the poration solution. In some embodiments, the disaccharide is about 0.05 M to about 5 M in the poration solution. In some embodiments, the disaccharide is about 0.1 M to about 3 M in the poration solution. In some embodiments, the disaccharide is about 0.1 M to about 1 M in the poration solution. In some embodiments, the disaccharide is about 0.2 M to about 0.6 M in the poration solution. In some embodiments, the disaccharide is about 0.4 M in the poration solution.

In some embodiments, the sugar is trehalose. In some embodiments, the trehalose is about 0.05 M to about 5 M in the composition comprising the population of cells. In some embodiments, the trehalose is about 0.1 M to about 3 M in the composition comprising the population of cells. In some embodiments, the trehalose is about 0.1 M to about 1 M in the composition comprising the population of cells. In some embodiments, the trehalose is about 0.2 M to about 0.6 M in the composition comprising the population of cells. In some embodiments, the trehalose is about 0.4 M in the composition comprising the population of cells.

In some embodiments, the trehalose is in the poration solution. In some embodiments, the trehalose e is about 0.05 M to about 5 M in the poration solution. In some embodiments, the trehalose is about 0.1 M to about 3 M in the poration solution. In some embodiments, the trehalose is about 0.1 M to about 1 M in the poration solution. In some embodiments, the trehalose is about 0.2 M to about 0.6 M in the poration solution. In some embodiments, the trehalose is about 0.4 M in the poration solution. In some embodiments, the composition further comprises about 0.1 M, about 0.2 M, about 0.3 M, about 0.4 M, about 0.5 M, about 0.6 M, about 0.7 M, about 0.8 M, about 0.9 M, or about 1.0 M trehalose. In some embodiments, the composition further comprises about 0.5 M trehalose.

In some embodiments, the methods of producing a population of lyophilized cells as described herein comprises a step wherein prior to the freezing, the population of cells is isolated (e.g., centrifuged or filtered) and resuspended in a poration solution. The poration solution comprises the sugar, e.g., trehalose in an aqueous solution. In some embodiments, the poration solution comprises sugar and water. In some embodiments, the poration solution comprises trehalose and water. In some embodiments, the population of cells is placed in the poration solution, and then subjected to thermal treatment. In some embodiments, the thermal treatment is performed as outlined by He et al., 2006, supra. In some embodiments, the thermal treatment includes, e.g., placing the population of cells in the poration solution, then cooling the composition for 1 to 60 minutes, e.g., 2 to 50 minutes, 3 to 40 minutes, 4 to 30 minutes, 5 to 20 minutes, 5 to 15 minutes, or about 10 minutes, followed by heating the composition for 1 to 60 minutes, e.g., 2 to 50 minutes, 3 to 40 minutes, 4 to 30 minutes, 5 to 20 minutes, 5 to 15 minutes. The cooling and heating can be repeated one, two, three, four, five, six, seven, eight, nine or ten times. In some embodiments, the cooling and heating can be repeated one, two, or three times. In some embodiments, the cooling was from −20° C. to a 10° C., or −10° C. to 10° C., or 0° C. to 10° C., or 2° C. to 8° C. In some embodiments, the warming was from 0° C. to 50° C., or 4° C. to 40° C., 8° C. to 40° C. or 20° C. to 40° C. In some embodiments, the difference between the cooling temperature and the warming temperature was greater than 4° C., greater than 6° C., greater than 8° C., greater than 10° C., greater than 15° C., or greater than 20° C.

Polysaccharides are polymeric carbohydrate molecules composed of long chains of monosaccharide units bound together by glycosidic linkages. Polysaccharide, as used herein, has at least four, more preferably at least five, most preferably at least ten, for instance at least fifty saccharide units, or at least 100 saccharide units, or at least 200 saccharide units. In some embodiments, the polysaccharide is primarily linear. In some embodiments, the polysaccharide can be branched. In some embodiments, the polysaccharide can be a polyglucan.

In some embodiments, the polysaccharide is dextran. Dextrans are polysaccharides with molecular weights greater than 100 Dalton, e.g., greater than 1,000 Dalton, which have a linear backbone of α-linked d-glucopyranosyl repeating units. Three classes of dextrans can be differentiated by their structural features. The pyranose ring structure contains five carbon atoms and one oxygen atom. Class 1 dextrans contain the α(1→6)-linked d-glucopyranosyl backbone modified with small side chains of d-glucose branches with α(1→2), α(1→3), and α(1→4)-linkage. The class 1 dextrans vary in their molecular weight, spatial arrangement, type and degree of branching, and length of branch chains, depending on the microbial producing strains and cultivation conditions. Isomaltose and isomaltotriose are oligosaccharides with the class 1 dextran backbone structure. Class 2 dextrans (alternans) contain a backbone structure of alternating α(1→3) and α(1→6)-linked d-glucopyranosyl units with α(1→3)-linked branches. Class 3 dextrans (mutans) have a backbone structure of consecutive α(1→3)-linked d-glucopyranosyl units with α(1→6)-linked branches.

In some embodiments, the polysaccharide is a pullulan, dextrin or dextran sulfate. Pullulans are structural polysaccharides primarily produced from starch by the fungus Aureobasidium pullulans. Pullulans are composed of repeating α(1→6)-linked maltotriose (D-glucopyranosyl-α(1→4)-D-glucopyranosyl-α(1→4)-D-glucose) units with the inclusion of occasional maltotetraose units. Diffusion-ordered NMR spectroscopy has been used to achieve a simple estimation of the molecular weight of pullulan. The solution properties of pullulan in water have been studied, and it was confirmed that pullulan molecules behave as random coils in aqueous solution.

Dextrins are composed of D-glucopyranosyl units but have shorter chain lengths than dextrans. They start with a single α(1→6) bond, but continue linearly with α(1→4)-linked D-glucopyranosyl units. Dextrins are usually mixtures derived from the hydrolysis of starch and have found widespread use in the food, paper, textile, and pharmaceutical industries.

Dextran sulfates are derived from dextran via sulfation. They have become indispensable components in many molecular biology techniques, including the transfer of large DNA fragments from agarose gels and rapid hybridization, precipitation procedures for the quantitation of high-density lipoprotein cholesterol, 24 and inhibition of virion binding to CD4+ cells.

The polysaccharides as used herein can be isolated from any organism known to produce them. In some embodiments, the polysaccharide can comprise various molecular weights. For example, in some embodiments, the polysaccharide has a molecular weight of about 1,000 Da to about 300,000 Da, about 5,000 Da to about 200,000, or about 10,000 Da to about 60,000 Da. In some embodiments, the polysaccharide has a molecular weight of about 30,000 Da to about 50,000 Da. In some embodiments, the polysaccharide has a molecular weight of about 40,000 Da.

In some embodiments, the dextran can comprise various molecular weights. For example, in some embodiments, the dextran has a molecular weight of about 1,000 Da to about 300,000 Da, about 5,000 Da to about 200,000, or about 10,000 Da to about 60,000 Da. In some embodiments, the dextran has a molecular weight of about 30,000 Da to about 50,000 Da. In some embodiments, the dextran has a molecular weight of about 40,000 Da.

The polysaccharide can be present at various concentrations in the composition comprising a population of cells. In some embodiments, the polysaccharide is about 0.1 mg/mL to about 100 mg/mL. In some embodiments, the polysaccharide is about 1 mg/mL to about 25 mg/mL. In some embodiments, the polysaccharide is about 5 mg/mL to about 15 mg/mL. In some embodiments, the polysaccharide is about 10 mg/mL.

The dextran can be present at various concentrations in the composition comprising a population of cells. In some embodiments, the dextran is about 0.1 mg/mL to about 100 mg/mL. In some embodiments, the dextran is about 1 mg/mL to about 25 mg/mL. In some embodiments, the dextran is about 5 mg/mL to about 15 mg/mL. In some embodiments, dextran is about 10 mg/mL.

In some embodiments, the lyophilization agent is free of dimethyl sulfoxide (DMSO). In some embodiments, the lyophilization agent is free of a non-cell permeable reagent. A non-cell permeable reagent does not move across the cell membrane. Non-cell permeable reagents include, for example, trehalose, sucrose, dextran, hydroxyethyl starch (HES), polyvinylpyrrolidone (PVP), and polyethylene oxide (PEO).

In some embodiments, the composition comprising a population of cells does not comprise DMSO, i.e., is free of DMSO. In some embodiments of the present disclosure, the methods of lyophilizing a population of cells as described herein does not include the addition of DMSO. In other words, in some embodiments, DMSO is not present or used in any step during the lyophilization process. In some embodiments, the concentration of DMSO in the composition comprising a population of cells is less than the conventional amount (e.g., 2-10%). In some embodiments, the concentration of DMSO in the composition comprising a population of cells is less than 1%, less than 0.8%, less than 0.5%, less than 0.2%, less than 0.1%, less than 0.05%, or less than 0.02%.

In some embodiments, the lyophilization agent further comprises thiobarbituric acid, polyvinylpyrrolidone, polyoxyethylene stearyl ether, cyclodextrin, hydroxyethyl starch, or combinations thereof.

In some embodiments, the tonicity of the composition can affect cell viability and attachment. “Tonicity” refers to the ability of an extracellular solution to make water move into or out of a cell by osmosis. Tonicity is related to a solution's osmolarity, i.e., the total concentration of all solutes in the solution. An “isotonic” solution refers to a solution that is the same osmolarity as the cell, and thus there is no net movement of water into or out of the cell. A “hypertonic” solution refers to a solution that is higher than the osmolarity of the cell, and thus water will move out of the cell into the extracellular solution. A “hypotonic” solution refers to a solution that is lower in osmolarity of the cell, and thus water will move into the cell from the extracellular solution. In some embodiments, the composition comprising the population of cells is an isotonic solution. In some embodiments, the composition comprising the cells is a hypertonic solution. In some embodiments, the tonicity of the composition facilitates the lyophilization process, protects the cells from damage or lysis, and/or increases cell viability.

In some embodiments, the aqueous component of the composition comprises buffer. Non-limiting examples of buffers include phosphate buffer, Tris buffer, HEPES buffer, acetate buffer, bicarbonate buffer, citrate buffer, tricine buffer, TES buffer, and the like. In some embodiments, the aqueous component of the composition comprises phosphate buffer, Tris buffer, acetate buffer, bicarbonate buffer, histidine buffer, citrate buffer, or combinations thereof.

In some embodiments, the aqueous component of the composition comprises cell culture medium. Non-limiting examples of cell culture medium include DMEM, MEM, RPMI 1640, EXPI293, OPTI-MEM, and STEMPRO from GIBCO, HYQ-RS from HYCLONE, X-VIVO and Hybridoma Serum-Free Culture Media from LONZA BIOWHITTAKER, and the like. In some embodiments, the medium is free of serum. In some embodiments, the medium is free of fetal bovine serum, bovine serum, or human serum. In some embodiments, the medium is a chemically defined medium. An exemplary chemically-defined medium may include a basal media (such as, e.g., DMEM, F12, or RPMI 1640), sugars such as dextrose and glucose, amino acids, vitamins, inorganic salts, buffers, antioxidants, growth factors and energy sources, recombinant serum albumin, chemically defined lipids, recombinant insulin and/or zinc, recombinant transferrin or iron, selenium, and/or antioxidant thiols such as 2-mercaptoethanol or 1-thioglycerol. In some embodiments, the cell culture medium comprises dextrose, glucose, amino acids, recombinant serum albumin, growth factors, or combinations thereof.

In some embodiments, the lyophilization process does not lyse or damage the cells. In some embodiments, the lyophilized cells are capable of being reconstituted and being viable. In the context of lyophilization, “reconstitution” refers to the process of rehydrating a lyophilized substance, e.g., lyophilized cells. In some embodiments, cells that are reconstituted after lyophilization are viable. In some embodiments, cells that are reconstituted after lyophilization are capable of performing the same cellular functions as a cell not subjected to lyophilization. In some embodiments, cells that are reconstituted after lyophilization are viable. In some embodiments, cells that are reconstituted after lyophilization retain the ability to adhere or attach to other cells. In some embodiments, cells that are reconstituted after lyophilization retain the ability to proliferate. In some embodiments, stem cells that are reconstituted after lyophilization retain the ability to differentiate. In some embodiments, cells that are reconstituted after lyophilization are suitable for use in cell therapy, e.g., introduction into a human or animal.

In some embodiments, the reconstitution of lyophilized cells comprises resuspending the lyophilized cells in a reconstitution agent to form a reconstituted composition. In the context of lyophilization, “reconstitution agent” refers to a substance that resuspends and rehydrates the lyophilized product. In some embodiments, the reconstitution agent comprises water. In some embodiments, the reconstitution agent comprises buffer. In some embodiments, the reconstitution agent comprises cell culture media. In some embodiments, the reconstitution agent comprises polyvinylpyrrolidone (PVP). In some embodiments, the reconstitution agent comprises trehalose. In some embodiments, the reconstitution agent comprises PVP, trehalose, or combinations thereof. In some embodiments, the reconstitution agent comprises phosphate buffer solution (PBS). In some embodiments, the reconstitution agent can be a solution known to maintain the viability of the cells.

In some embodiments, the reconstitution agent comprises PVP, and the PVP is about 1% to about 30% of the reconstituted composition. In some embodiments, the PVP is about 5% to about 20% of the reconstituted composition. In some embodiments, the PVP is about 10% to about 15% of the reconstituted composition. In some embodiments, the PVP is about 12% of the reconstituted composition. In some embodiments, the reconstitution agent comprises trehalose, and the trehalose is about 0.01 M to about 0.05 M in the reconstituted composition. In some embodiments, the trehalose is about 0.02 M to about 0.04 M in the reconstituted composition. In some embodiments, the trehalose is about 0.02 M of the reconstituted composition. In some embodiments, the reconstitution agent comprises about 10% to about 15% PVP, about 0.01 M to about 0.05 M trehalose, and phosphate buffer solution. In some embodiments, the reconstitution agent comprises about 12% PVP, about 0.02 M trehalose, and phosphate buffer solution.

In some embodiments, the lyophilized cells can be stored for a period of time and then reconstituted. In some embodiments, resuspending the lyophilized cell occurs greater than two hours after the removing the aqueous component, greater than one day after the removing the aqueous component, greater than one week after the removing the aqueous component or greater than one month after the removing the aqueous component.

As used herein, the terms “store” or “storage,” when referring to samples or compositions such as lyophilized cells, refer to subjecting the lyophilized composition to temperatures of about −80° C. to about 40° C., or about −60° C. to about 35° C., or about −20° C. to about 30° C., or about 0° C. to about 29° C., or about 4° C. to about 28° C., or about 10° C. to about 27° C., or about 15° C. to about 26° C., or about 20° C. to about 25° C., or about 2° C. to about 8° C., for a period of time between about 1 hour to about 1 month, or about 12 hours to about 15 days, or about 1 day to about 10 days, or about 3 days to about 8 days, or about 5 days to 6 days, or about 2 hours, or about 1 day, or about 1 week, or about 1 month, or greater than about 1 month. In some embodiments, the terms “store” or “storage” refers to subjecting a sample or composition to temperature of greater than about −20° C. for greater than 2 days. In some embodiments, the terms “store” or “storage” refer to subjecting a sample or composition to temperature of about 20° C. to about 25° C. for greater than 1 week. In some embodiments, the terms “store” or “storage” refer to subjecting a sample or composition to temperatures of about 20° C. to about 25° C. for between about 1 week and about 1 month.

In some embodiments, the terms “store” or “storage” refer to subjecting a sample or composition to temperature of about 2° C. to about 8° C. for greater than 1 week. In some embodiments, the terms “store” or “storage” refer to subjecting a sample or composition to temperatures of about 2° C. to about 8° C. for between about 1 week and about 1 month.

In some embodiments, the terms “storage time” or “storage period” refer to the period of time after removing the aqueous component from the composition and resuspending the lyophilized cells.

In some embodiments, the resuspending the lyophilized cell occurs greater than 2 hours after the removing the aqueous component. In some embodiments, the resuspending the lyophilized cell occurs greater than 1 day after the removing the aqueous component. In some embodiments, the resuspending the lyophilized cell occurs greater than 1 week after the removing the aqueous component. In some embodiments, the resuspending the lyophilized cell occurs greater than 1 month after the removing the aqueous component. In some embodiments, the resuspending the lyophilized cell occurs greater than about 1 hour, 2 hours, 6 hours, 12 hours, 1 day, 2 days, 3 days, 4 days, 5 days, 6 days, 1 week, 2 weeks, 3 weeks, 1 month, 3 months, 6 months, 1 year, 2 years, 3 years, 4 years, or 5 years after the removing the aqueous component. In some embodiments, the storage time of the lyophilized cells does not substantially decrease the viability of the cells.

In some embodiments, the lyophilized cells are stored at about −20° C. to about 30° C. prior to the resuspending. In some embodiments, the lyophilized cells are stored at about 4° C. to about 28° C. prior to the resuspending. In some embodiments, the lyophilized cells are stored at about 10° C. to about 27° C. prior to the resuspending. In some embodiments, the lyophilized cells are stored at about 15° C. to about 26° C. prior to the resuspending. In some embodiments, the lyophilized cells are stored at about 20° C. to about 25° C. prior to the resuspending. In some embodiments, the lyophilized cells are stored at greater than about −20° C., about −15° C., about −10° C., about −5° C., about 0° C., about 4° C., about 10° C., about 15° C., about 20° C., or about 25° C. prior to the resuspending. In some embodiments, the temperature at which the lyophilized cells are stored does not substantially decrease the viability of the cells.

A “viable” cell refers to an alive and functioning cell, e.g., a cell that is capable of survival and performing normal cellular functions. In some embodiments, a normal cellular function of a cell is adhesion or attachment to other cells. “Cell adhesion” or “cell attachment” refers to a process by which cells interact and attach to neighboring cells and/or to the surface of their growth containers through interactions of surface proteins. Cells not capable of attaching may be unable to proliferate or perform normal cellular functions. For example, a stem cell that is not capable of attaching may be unable to differentiate. In another example, a cell that is introduced into a patient for cell therapy, but is unable to attach, may not provide therapeutic benefits because the cell is not capable of performing normal functions in the patient. Thus, in some embodiments, a viable cell is a cell that is capable of attachment. In some embodiments, a viable cell is a cell that is capable of proliferation. In some embodiments, a viable stem cell is a stem cell that is capable of differentiation.

In some embodiments, viability of a cell is determined, e.g., by measuring the redox potential and/or metabolic activity of the cell population. The redox potential and/or metabolic activity of a cell population can be measured by reagents such as resazurin, a component of the reagents ALAMARBLUE® and PRESTOBLUE®; MTT (3-(4,5-dimethylthiazol-2-yl)-2,5-diphenyl tetrazolium bromide), used in the VYBRANT MTT Cell Viability Assay; or XTT (2,3-bis-(2-methoxy-4-nitro-5-sulfophenyl)-2H-tetrazolium-5-carboxanilide), used in the CYQUANT XTT Cell Viability Assay. In some embodiments, viability of a cell is determined by measuring or inspecting the integrity of the cellular membrane, for example, using microscopy or flow cytometry. In some embodiments, viability of a cell is measured after a cell is lyophilized, then reconstituted.

Additional examples of cell viability assays are described in, e.g., Riss et al., “Cell Viability Assays,” 2013 May 1 [Updated 2016 Jul. 1]. In: Sittampalam G S, Coussens N P, Brimacombe K, et al., editors. Assay Guidance Manual [Internet]. Bethesda (M D): Eli Lilly & Company and the National Center for Advancing Translational Sciences; 2004. Available from: www.ncbi.nlm.nih.gov/books/NBK144065.

The skilled artisan can appreciate that when discussing viability of cells, i.e., a population of cells, that not 100% of the cells will be viable. In some embodiments, a viable cell population comprises at least 1% viable cells. In some embodiments, a viable cell population comprises at least 5% viable cells. In some embodiments, a viable cell population comprises at least 10% viable cells. In some embodiments, a viable cell population comprises at least 15% viable cells. In some embodiments, a viable cell population comprises at least 20% viable cells. In some embodiments, a viable cell population comprises at least 30% viable cells. In some embodiments, a viable cell population comprises at least 40% viable cells. In some embodiments, a viable cell population comprises at least 50% viable cells. In some embodiments, a viable cell population comprises at least 60% viable cells. In some embodiments, a viable cell population comprises at least 70% viable cells. In some embodiments, a viable cell population comprises at least 80% viable cells. In some embodiments, a viable cell population comprises at least 90% viable cells. In some embodiments, a viable cell population comprises about 1% viable cells to about 99% viable cells, 5% viable cells to about 99% viable cells, 10% viable cells to about 99% viable cells, 20% viable cells to about 99% viable cells, about 30% viable cells to about 99% viable cells, about 40% viable cells to about 99% viable cells, about 50% viable cells to about 99% viable cells, about 60% viable cells to about 99% viable cells, about 70% viable cells to about 99% viable cells, about 80% viable cells to about 99% viable cells, or about 90% viable cells to about 99% viable cells.

In some embodiments, a viable cell population comprises at least 1% of cells capable of attachment. In some embodiments, a viable cell population comprises at least 5% of cells capable of attachment. In some embodiments, a viable cell population comprises at least 10% of cells capable of attachment. In some embodiments, a viable cell population comprises at least 20% of cells capable of attachment. In some embodiments, a viable cell population comprises at least 30% of cells capable of attachment. In some embodiments, a viable cell population comprises at least 40% of cells capable of attachment. In some embodiments, a viable cell population comprises at least 50% of cells capable of attachment. In some embodiments, a viable cell population comprises at least 60% of cells capable of attachment. In some embodiments, a viable cell population comprises at least 70% of cells capable of attachment. In some embodiments, a viable cell population comprises at least 80% of cells capable of attachment. In some embodiments, a viable cell population comprises at least 90% of cells capable of attachment. In some embodiments, a viable cell population comprises about 1% to about 99% cells capable of attachment, 5% to about 99% cells capable of attachment, 10% to about 99% cells capable of attachment, 20% to about 99% cells capable of attachment, about 30% to about 99% cells capable of attachment, about 40% to about 99% cells capable of attachment, about 50% to about 99% cells capable of attachment, about 60% to about 99% cells capable of attachment, about 70% to about 99% cells capable of attachment, about 80% to about 99% cells capable of attachment, or about 90% to about 99% cells capable of attachment.

In some embodiments, the reconstituted cells are viable. Methods of measuring viability of cells are described herein. In some embodiments, cell viability is measured by staining and detecting an intact cell membrane. In some embodiments, cell viability is measured by assessing metabolic activity, e.g., by using the ALAMARBLUE®, MTT, or XTT tests. In some embodiments, at least 1% of the cells in the reconstituted composition are viable. In some embodiments, at least 5% of the cells in the reconstituted composition are viable. In some embodiments, at least 10% of the cells in the reconstituted composition are viable. In some embodiments, at least 20% of the cells in the reconstituted composition are viable. In some embodiments, at least 30% of the cells in the reconstituted composition are viable. In some embodiments, at least 40% of the cells in the reconstituted composition are viable. In some embodiments, at least 50% of the cells in the reconstituted composition are viable. In some embodiments, at least 60% of the cells in the reconstituted composition are viable. In some embodiments, at least 70% of the cells in the reconstituted composition are viable. In some embodiments, at least 80% of the cells in the reconstituted composition are viable. In some embodiments, at least 90% of the cells in the reconstituted composition are viable.

In some embodiments, the reconstituted cells are capable of attachment, as measured by ALAMARBLUE® test. As described herein, the ALAMARBLUE® test assesses metabolic activity of the cells, which can be an indicator of cell attachment. In some embodiments, at least 20% of the cells in the reconstituted composition attach as measured by ALAMARBLUE® test. In some embodiments, at least 30% of the cells in the reconstituted composition attach as measured by ALAMARBLUE® test. In some embodiments, at least 40% of the cells in the reconstituted composition attach as measured by ALAMARBLUE® test. In some embodiments, at least 50% of the cells in the reconstituted composition attach as measured by ALAMARBLUE® test. In some embodiments, at least 60% of the cells in the reconstituted composition attach as measured by ALAMARBLUE® test. In some embodiments, at least 70% of the cells in the reconstituted composition attach as measured by ALAMARBLUE® test. In some embodiments, at least 80% of the cells in the reconstituted composition attach as measured by ALAMARBLUE® test. In some embodiments, at least 90% of the cells in the reconstituted composition attach as measured by ALAMARBLUE® test.

In some embodiments, the present disclosure provides a method of detecting non-cellular particles in a cell composition. Detection of non-cellular particles may be useful when evaluating the quality of a cell composition for therapeutic applications, e.g., a cell population introduced into a human patient. In some embodiments, the method of detecting non-cellular particles comprises capturing an image of a cell composition with an imaging flow cytometer and analyzing the image to distinguish between cells and non-cellular particles based on one or more of width, diameter, circularity, intensity, compactness, convexity, and edge gradient of the captured image. In some embodiments, the method further comprises separating the cells from the non-cellular particles.

In some embodiments, the disclosure provides a method of producing a population of lyophilized cells in a matrix without the use of a cryoprotectant. In some embodiments, the disclosure provides a method of producing a population of lyophilized cells in a matrix without the use of DMSO. In some embodiments, the disclosure provides a method of producing a population of lyophilized cells in a matrix without the use of a polyol. In some embodiments, the disclosure provides a method of producing a population of lyophilized cells in a matrix without the use of a sugar. In some embodiments, the disclosure provides a method of producing a population of lyophilized cells in a matrix without the use of a polysaccharide.

In some embodiments, the disclosure provides a method of producing a population of lyophilized cells, the method comprising: (a) freezing a composition comprising a population of cells and an aqueous component in a hydrogel matrix; and (b) removing at least about 90% of the aqueous component from the frozen composition to produce the population of lyophilized cells.

In some embodiments, the disclosure provides a method of producing a population of reconstituted viable cells, comprising: (a) freezing a composition comprising a population of cells and an aqueous component in a hydrogel matrix; (b) removing at least about 90% of the aqueous component from the frozen composition to produce the population of lyophilized cells, and (c) resuspending the population of lyophilized cells in a reconstitution agent to form a reconstituted composition, wherein at least about 1% of the cells are viable. In some embodiments, the hydrogel is a biocompatible hydrogel. In some embodiments, the hydrogel is a hyaluronan gel, alginate gel or collagen gel. In some embodiments, the population of cells in (a) is suspended in the hydrogel.

In some embodiments, the disclosure provides a method of producing a population of lyophilized cells in a matrix with the use of a cryoprotectant. For example, the disclosure provides a method comprising (a) freezing a composition comprising a population of cells and an aqueous component in a hydrogel matrix; and (b) removing at least about 90% of the aqueous component from the frozen composition to produce the population of lyophilized cells, wherein the composition comprising the population of cells in (a) further comprises a polyol, a sugar, a polysaccharide, or combinations thereof. In some embodiments, the population of cells of (a) is about 1×10⁴ cells per mL to 1×10⁶ cells per mL. In some embodiments, the sugar is a disaccharide. In some embodiments, the disaccharide is selected from the group consisting of maltose, lactose, sucrose, lactulose, trehalose, cellobiose, isomaltose, melibiose, and gentiobiose. In some embodiments, the sugar is trehalose. In some embodiments, the polyol is selected from glycerol, propylene glycol and ethylene glycol. In some embodiments, the polyol is about 0.05 M to about 5 M. In some embodiments, the polyol is glycerol. In some embodiments, the glycerol is about 0.01 M to about 10 M in the composition. In some embodiments, the polysaccharide is about 1 mg/mL to about 25 mg/mL. In some embodiments, polysaccharide is a polyglucan. In some embodiments, polyglucan is a dextran. In some embodiments, the dextran has a molecular weight of about 10,000 Da to about 60,000 Da. In some embodiments, the composition of (a) comprising cryoprotectants is free of DMSO.

In some embodiments, the population of cells can be propagated/grown in the hydrogel matrix, and then subjected to the lyophilization process as described herein. Thus, in some embodiments, the disclosure provides for increased efficiency, whereby the cells do not have to be isolated prior to lyophilization.

All references cited herein, including patents, patent applications, papers, textbooks and the like, and the references cited therein, to the extent that they are not already, are hereby incorporated herein by reference in their entirety.

### EXAMPLES

### Examples Overview

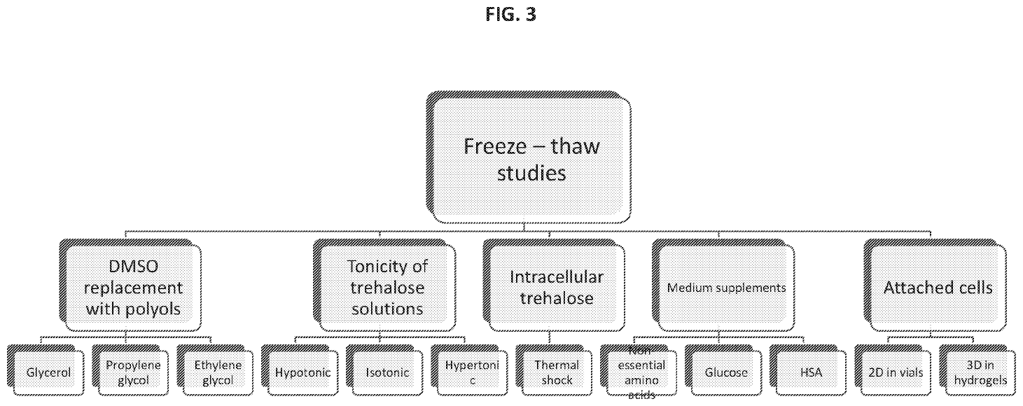

Various assays were conducted in an attempt to improve survival of cells during freezing using SK-N-AS cancer cell line as a model cell line in different formulations and growth matrices. The assays included the following:
Evaluating alternative cryoprotective agents.
Assessing the impact of tonicity on cell survival.
Testing the effect of cell medium supplements on cell survival.
Introducing trehalose intracellularly via thermal shock.
Exploring the use of 2D and 3D cell attachment for lyophilization.
   A brief overview of these assays is outlined in FIG. 3.

### Materials and Methods

Cell Line
Human neuroblastoma cells SK-N-AS (ATCC® CRL-2137™)

Substances for Cell Culturing
Phosphate Buffer Saline (PBS), pH 7.4 (GIBCO™, Fisher Scientific)
Dulbecco's Modified Eagle Medium (DMEM), high glucose, pyruvate (GIBCO™, Fisher Scientific)
Trypsin-EDTA (0.25%) with Phenol red (GIBCO™, Fisher Scientific) Fetal Bovine Serum (FBS), qualified, heat inactivated, E.U.-approved, South America Origin (GIBCO™, Fisher Scientific)
MEM Non-essential Amino Acid Solution—NEAA, 100× conc. (Sigma-Aldrich)
Penicillin-Streptomycin—Pen/Strep, 100× conc. (Sigma-Aldrich)

Excipients
Glycerol, purity ≥99% (Sigma-Aldrich)
Propylene glycol, purity ≥99.5% (Sigma-Aldrich)
Ethylene glycol, purity ≥99% (Sigma-Aldrich)
Dimethyl Sulfoxide—DMSO, purity ≥99.8% (CARL ROTH®)
Trehalose dihydrate (Pfanstiehl)
Dextran MW 40 000 (MP BIOMEDICALS™)
Hyaluronic acid sodium salt from Streptococcus equi—mol wt ˜1.5-1.8×10E6 Da (Sigma-Aldrich)
Collagen from calf skin (Sigma-Aldrich)
Brij L23 (Sigma-Aldrich)
PEG 400 (Sigma-Aldrich)
PEG 6000 (Sigma-Aldrich)
Hydroxyethyl starch—HES (Sigma-Aldrich)

Laboratory Equipment
75 cm² and 225 cm² cell culturing flasks NUNC™ EASYFLASK™ (Thermo Fisher Scientific)
96-well plates NUNC™ F96 MICROWELL™ (Thermo Fisher Scientific)

Bioassay Consumables
VIAL-CASSETTE™ for NUCLEOCOUNTER® NC-200™ (Chemometec)
ALAMARBLUE® reagent (Thermo Fisher Scientific)

Instruments
Suction instrument VACUBOY® (Integra)
Light microscope—CKX53 (Olympus)
NUCLEOCOUNTER® NC-200™ (Chemometec)
Pilot scale freeze dryer LyoStar 3 (SP Scientific)

### Example 1

### Methods

### SK-N-AS Medium Preparation

Medium for SK-N-AS cell line was prepared by first thawing FBS and Pen/Strep and warming them together with DMEM solution and NEAA to 37° C. in a water bath. Next 50 mL FBS, 5 mL NEAA and 5 mL Pen/Strep was pipetted into 500 mL bottle of DMEM. Solution was gently mixed and stored at 2-8° C.

### Thawing Cells from Cell Bank

First the cell medium was warmed up to 37° C. and then 9 mL were pipetted into 15 mL Falcon tube. Cells, that were stored in liquid nitrogen, were thawed in a water bath at 37° C. and then transferred (1 mL) into prepared Falcon tube with medium. This cell suspension was then centrifuged at 140 G for 4 minutes.

The supernatant was aspirated and the cell pellet re-suspended in 6 mL of medium. The suspension was then transferred into a 75 cm² flask. The cells were incubated at 37° C. and 5% CO₂. Next day the old medium was aspirated with use of VACUBOY® and replaced with 12 mL of fresh medium.

### Splitting the Cells

Splitting of cells was performed by first aspirating the medium from the flask and then washing the cells with 8mL of PBS. After aspirating PBS, 2 mL of trypsin (0.25%) was pipetted and incubated at 37° C. for 3 minutes. After the cells were loose, 8mL of medium was added.

The suspension was mixed with a serological pipette and transferred into a 15 mL falcon tube to harvest the cells. The suspension was centrifuged at 140 G for 4 minutes. After that, the supernatant was aspirated and the cell pellet re-suspended with 6mL of medium. 0.5 mL of suspension was then pipetted into each of six prepared 75 cm² flasks with 12 mL of medium and incubated them at 37° C. and 5% CO₂.

### Cell Passaging

Cells were harvested from 75 cm² flasks into falcon tubes and centrifuged at 140 G for 4 minutes. The supernatant from falcon tubes was aspirated after centrifugation and pellets were re-suspended with 3 mL of medium. Suspensions were then transferred into 40 mL medium in 225 cm² flasks and incubated at 37° C. and 5% CO₂.

### Results

### Cell Counting and Viability Assay: 4′,6-diamidino-2-phenylindole (DAPI) and Acridine Orange Ataining with NUCLEOCOUNTER® NC-200

200 μL of cell suspension was pipetted into 1.5 mL Eppendorf tube and diluted with medium. Diluted suspension was then pipetted with VIAL-CASSETTE™ and inserted into the NUCLEOCOUNTER® for measurement.

### ALAMARBLUE® Assay

The cells were plated on a 96-well plate in a density of 2.25×10⁵ cells/cm² and diluted with medium to the final volume of 200 μL inside the well. The cells were then incubated overnight at 37° C. and 5% CO₂ so they attached to the wells. Cells were stained with 10 μL ALAMARBLUE® reagent per 100 μL of medium in the well and incubated for 4 h. Same procedure was applied for a blank, where 200 μL of medium was pipetted into the well. After that, 100 μL of solution above the cells was pipetted into a black non-transparent 96-well plate and fluorescence was measured with SPECTRAMAX® plate reader (excitation λ=550 nm, emission λ=590 nm).

For 3D cell cultures in hydrogel inserts, the assay was performed by addition of ALAMARBLUE® into the well in the same concentration as above and incubated for 4 h. 100 μL of stained solution was then pipetted from the well into the non-transparent 96-well plate for fluorescence measurement. A hydrogel without cells served as a blank. Same procedure was applied to 3D cell cultures inside the vials, where the stained solution was above the hydrogel layer.

### Example 2—Freeze-Thaw Experiments

### SK-N-AS Growth Curve

Before beginning with further experiments, an optimal cell density, for the cells to grow on plates was investigated. A growth curve was generated. This was done by plating the cells at different densities on a 96-well plate and measuring the density periodically. The best performing initial cell density was then chosen for future experiments.

Cell suspension was pipetted on a 96-well plate, so the cell densities were in different wells: 0.30×10⁵ cells/cm², 1.53×10⁵ cells/cm², 2.25×10⁵ cells/cm², 3.00×10⁵ cells/cm², 4.50×10⁵ cells/cm² and 7.50×10⁵ cells/cm². Each density was plated in 6 replicates. ALAMARBLUE® assay was performed after incubation at 37° C. and 5% CO₂ for 1, 2, 3, 4 and 7 days. A growth curve was generated and different densities were evaluated according to respective growth rate.

### Cell Freeze—Thawing

Formulations were prepared in 50 mL Falcon tubes and subsequently sterile filtered under the bench with 0.22 μm syringe filter and a syringe. Sterile formulations were then stored at 2-8° C. until further use. 0.5 mL of cell suspension in either PBS or medium at a concentration of 20*10⁶ cells/mL was mixed with 0.5 mL of pre-warmed sterile formulation. Vials were then stoppered and stored in a −80° C. freezer for minimum overnight.

Subsequently, the vials were taken from the −80° C. freezer and rapidly thawed in the water bath at 37° C. Immediately after thawing, 0.1 mL of cell suspension was 2× diluted with medium and counted with NucleoCounter to obtain cell viability [%] and concentration inside the vial. Suspension was then plated on a 96-well plate so the cell density was 2.25×10⁵ cells/cm² (growth area in one well is 0.32 cm²). Calculation of the volume (V) of cell suspension is according to following equation:

$$V ( suspension ) = \frac{2.25 \times 1 {0}^{5} cells / {cm}^{2} \times 0.32 {cm}^{2}}{Con c . ( suspension ) [ cell / mL ]}$$

Calculated volume was diluted inside of the well with cell growth medium, so that the final volume was 0.2 mL. 96-well plate with samples was incubated overnight in the incubator and ALAMARBLUE® assay was performed the morning after (if not stated differently in the results section).

### Example 3—Introduction of Trehalose Into Cells Via Thermal Treatment

Protocol for thermal treatment of cells was adapted from the research done by He et al. (He, X., et al., Cell Preservation Technology 4, 178-187 (2006)). Cells were harvested in a falcon tube, counted, spun down with the centrifuge and resuspended in poration solution (0.4M trehalose solution prepared in cell medium:water=1:2). Untreated cells were subsequently plated onto 96-well plate. Cell suspension was divided into 15 mL falcon tubes with 5.5 mL suspension in each tube. Cells from first series of tubes were immediately after plated into 96-well plate, spun down with the centrifuge, resuspended in formulations to be tested and frozen at −80° C. Other Falcon tubes went through thermal treatment program according to Table 1. After alternating between the cooling and heating process for 20 and 60 minutes, the cells were plated, reformulated and frozen as previously described.

TABLE 1
Thermal treatment program

| | | |
|---|---|---|
| Process | Temperature | Time |

| | | | |
|---|---|---|---|
| Cooling | 0° C. | (ice bath) | 10 minutes |
| Heating | 39° C. | (water bath) | 10 minutes |

Plated cells were incubated overnight and the next day ALAMARBLUE® assay was performed. Frozen cells were thawed the next day, counted with NUCLEOCOUNTER® and plated. After overnight incubation the ALAMARBLUE® assay was performed.

### Example 4—2D and 3D Attached Cells

### 2D Cell Culture in Glass Vials

Glass vials first needed to be coated with collagen. Two protocols were tested for this purpose (Table 2). After preparing two dilutions of rat tail collagen: 50 and 500 μg/mL, 0.2 mL of each was pipetted into respective 6R vials. Vials were incubated according to Table 2. Solution was carefully aspirated and vials were washed with PBS. Cells were then harvested and seeded on the coated vials with the cell density of 2.25×10⁵ cells/cm².

TABLE 2
Collagen coating protocols

| Protocol | Collagen density | Incubation | PBS washing |
|---|---|---|---|
| A | 10 μL/cm² | 1 h at room temperature | 3× |
| B | 100 μL/cm² | 30 min at 37° C. | 1× |

### 3D Cell Culture in Hydrogel Inserts

Cells were encapsulated in hydrogels using CORNING® Transwell inserts. Various hydrogels based on hyaluronan were tested: hyaluronic acid, HYSTEM™, HYSTEM-C™ and HYSTEM-HP™. HYSTEM™ gels were prepared according to the manual enclosed to the product. Before the gelation was fully completed, the cells were suspended inside the matrix and 0.1 mL of hydrogel was subsequently pipetted in the inserts on a 24-well plate. Inserts were incubated at 37° C. for one hour, so the gelation was fully finished. 1.8 mL of medium was added inside the wells with the inserts in a manner, that did not disturb the hydrogels and incubated in the incubator. Medium was changed every 2-3 days.

### 3D Cell Culture in Glass Vials

After gel preparation that followed the same procedure as for the gel inserts, the hydrogel cell suspension was pipetted inside the glass vials and left for an hour inside the incubator, for the gelation to be finished. 1.5 mL of medium was then pipetted on top of the hydrogel, the vial was half stoppered with a stopper and incubated inside the incubator. Medium was changed every 2-3 days, by first tilting the vial, aspirating the old medium and adding the fresh one.

### Example 5—Freeze Drying

Freeze drying was performed with the SP Scientific's LyoStar 3 pilot scale freeze dryer with 0.4 m² of shelf surface area. Vials were filled and loaded on a tray under the biosafety bench and then loaded in the freeze dryer. Vials were left on the tray inside the lyophilizer, to avoid any spillage of biomaterial inside the chamber in case of a vial breakage.

Employed conservative lyophilization programs (Table 3 and Table 4) included freezing and primary drying steps. Since it is known from literature, that complete elimination of water and higher temperatures employed during secondary drying negatively impact the cell survival during process, the step was omitted. The product temperature was monitored by including a thermocouple inside the vial, so that its tip touched the bottom of the vial. After unloading of the vials from the lyophilizer, they were visually inspected. Based on the cake appearance, the formulation was categorized either as lyophilizable or as not lyophilizable.

TABLE 3
Freeze-drying program employed of cells suspension samples.

| | Shelf temperature | Chamber pressure | Ramp | Hold |
|---|---|---|---|---|
| Step | setpoint [° C.] | setpoint [mTorr] | time [min] | time [min] |
| Freezing | −50 | — | Max speed | 360 |
| Drying | −50 | 30 | 0 | 420 |
| Drying | −35 | 30 | 30 | 3600 |

TABLE 4
Freeze-drying program employed for hydrogel samples.

| | Shelf temperature | Chamber pressure | Ramp | Hold |
|---|---|---|---|---|
| Step | setpoint [° C.] | setpoint [mTorr] | time [min] | time [min] |
| Freezing | −60 | — | Max speed | 360 |
| Drying | −50 | 20 | 20 | 1440 |
| Drying | −35 | 20 | 30 | 2000 |

### Results and Discussion

### Method Establishment

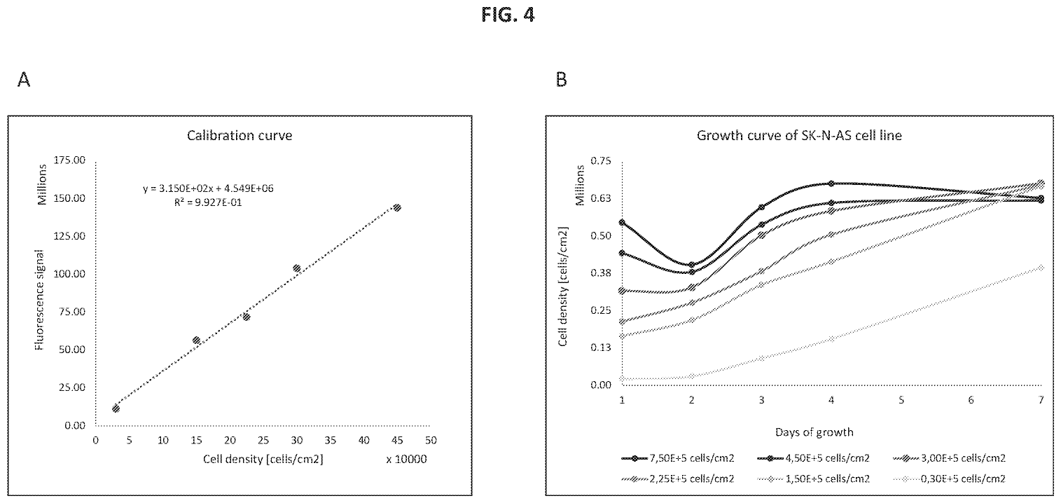

Two method for measuring the cell viability were tested—DAPI/acridine orange staining and ALAMARBLUE® assay. To determine cell density of viable plated cells from fluorescence signal, produced with the latter method, a calibration curve was generated (FIG. 4A). The relationship between fluorescence and cell densities from 3×10⁴ cells/cm² and 45×10⁴ cells/cm² was linear with R²=0.9927 (fluorescence measured at five different cell densities). Simultaneously, the best cell density was then chosen for plating in subsequent experiments, from the rates of linear growth and shortest lag periods. Based on the results illustrated in FIG. 4B, the cell density was 2.25×10⁵ cells/cm² was selected for future assays.

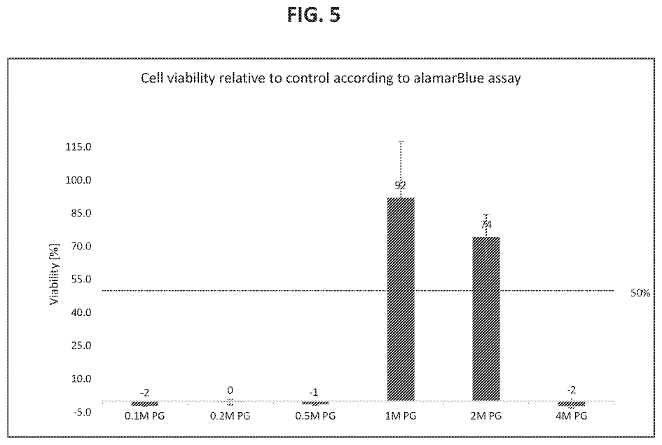

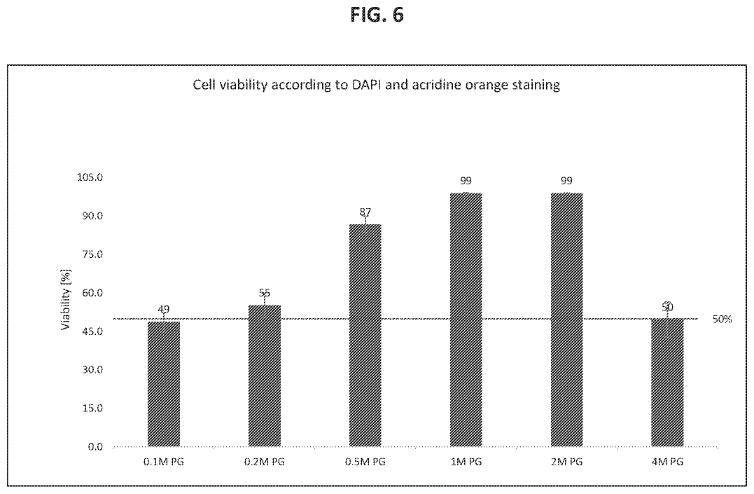

Next, we compared two methods for determining viability and noticed a large discrepancy in obtained results (FIG. 5 and FIG. 6). The reason for this is the difference in underlying principles of viability assessment of both methods. As previously, DAPI/acridine orange staining is performed using NUCLEOCOUNTER®, which is an integrated fluorescence microscope with dual fluorescence channels. One is for staining with acridine orange and the other with DAPI dye. They both bind to DNA and label cellular nuclei with high specificity. Acridine orange can permeate the cell membrane and is used for cell detection, while DAPI cannot permeate the membrane and binds only to nuclei of dead cells with disrupted membrane. Thus, the cell viability according to the DAPI/acridine orange method, is mostly based on the integrity of cell membranes. Dyes are fluorescent and therefore visible when using fluorescence filters in microscope, while cell debris and other particles without DNA are not visible. The software then identifies and analyses the individual cells (even within smaller aggregates) and calculates the viability, by comparing the ratio of signal with Acridine Orange and DAPI staining (according to equation below).

$$\% viability ( NUCLEOCOUNTER ® ) = \frac{{c}_{t} - {c}_{d}}{{c}_{t}} \times 100 \%$$

$$( {c}_{t} = total concentration of cells , {c}_{d} = concentration of dead cells )$$

ALAMARBLUE® on the other hand, contains a reagent called resazurin. This is a non-toxic, blue, weakly fluorescent dye, which can permeate cell membranes. Resazurin undergoes a metabolic reduction inside of the cell to a form called resorufin, which is pink and highly fluorescent. The intensity of fluorescence is proportional to the amount of cells that are able to metabolize resazurin and are thus considered viable.

DAPI/acridine orange staining was performed directly after thawing the vials, and ALAMARBLUE® assay was performed after cell plating and incubation overnight. The reason for this is, that for the cells must be attached for the metabolic assay. The results of DAPI/acridine testing suggested an almost 100% viability after FT of cells in formulations with 1M and 2M propylene glycol and viabilities of around 50% in formulations with 0.1M and 0.2M propylene glycol. In contrast, the results of metabolic assay show much lower cell viability of treated cells relative to untreated cells.

Not all the cells, which do not have a disrupted membrane are necessarily also viable and it is far more likely, that the cells with a functioning metabolism are viable. Due to this, the ALAMARBLUE® assay was chosen as a method for cell survival evaluation. In further experiments the cell density of plated cells is used as a measure of viability of the cells, which could be plated on 96-well plates. For cells, which were encapsulated in hydrogel scaffolds, the fluorescence signal produced by metabolising the ALAMARBLUE® reagent, was used as an indicator of cell viability.

### Freeze-Thaw Experiments

In order for cells to survive the process of freeze drying, they first need to go through the freezing step. Research work was therefore firstly focused on screening of excipients and their ability to preserve the cell viability after freeze-thawing.

### DMSO Replacement

Due to its toxicity, it is desirable to reduce or eliminate DMSO from formulations. Polyols such as glycerol and propylene glycol have lower toxicity and are like DMSO, membrane permeable substances. Their higher tonicity, therefore, doesn't put as high osmotic pressure on the cell membrane, as some membrane non-permeable substances would (trehalose for example). Three different polyol formulations were tested in different concentrations.

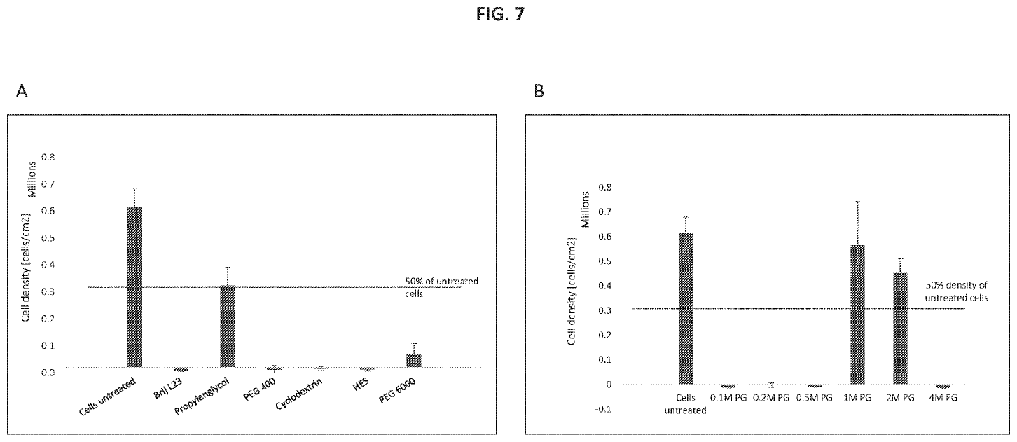

In search of alternative cryoprotectant, a series of excipients, viz. Brij L23, propylene glycol, PEG 400, cyclodextrin, HES and PEG 6000, was screened at the same concentrations of 5% (v/v) in cell medium. FIG. 7A. Our internal data has already established that 5% DMSO is optimal cryoprotectant to achieve over 80% of cell viability compared to the untreated control. The excipients were chosen based on their safety profile and chemical properties, which could potentially lead to cell protection during freezing. Relative viabilities to control—untreated cells, that did not undergo freeze thawing, are illustrated in FIG. 7A. Viability data suggested that propylene glycol was efficient in protecting the cells compared to other tested excipients at 5% concentration. To further optimise the concentration of propylene glycol and to find further potential cryoprotectants, other polyols were tested in separate experiments. Results of the screening are shown in FIG. 7B and they demonstrate, that 1M concentration of propylene glycol is the most favourable one.

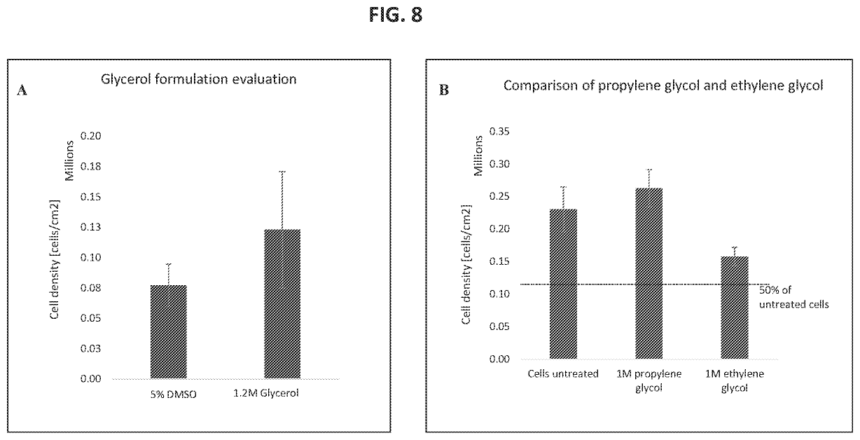

FIG. 8A and FIG. 8B show the evaluation of results of glycerol and ethylene glycol as cryoprotectants. FIG. 8A suggests that glycerol is not significantly worse than 5% DMSO formulation in medium. FIG. 8B shows that densities of plated cells after thawing in 1M propylene glycol and 1M ethylene glycol are not significantly different. One-way analysis of variance (ANOVA) and subsequent two-way t-test for all pairs proved that ethylene glycol formulation in medium is significantly worse than propylene glycol (P<0.05). In addition, ethylene glycol also has some toxicological concerns for human use.

### Tonicity of Formulations

Intracellular ice formation is undesirable due to its expansion and high risk of piercing the cellular membrane. Hence, dispersing the cells in hypertonic formulation before freezing might show better post-thaw viabilities due to water exiting the cells, which leads to less intracellular ice. This hypothesis was tested by performing freeze-thaw experiments with hypo-iso—and hypertonic formulations of trehalose, which is membrane non-permeable substance. Trehalose is also a known cryoprotectant and a lyoprotectant, especially for protein molecules. A benefit of trehalose is also, that it increases the Tg′ of the formulation, which allows higher temperatures of primary drying during lyophilization to be used. Since the polyols have a very low Tg, this increase via addition of trehalose could be vital for the feasibility of the process.

The polyol used for this part of the study was glycerol, which showed good cryprotective ability and has a lower glass transition temperature than propylene glycol. However, the same principles may apply to formulations with other polyols.

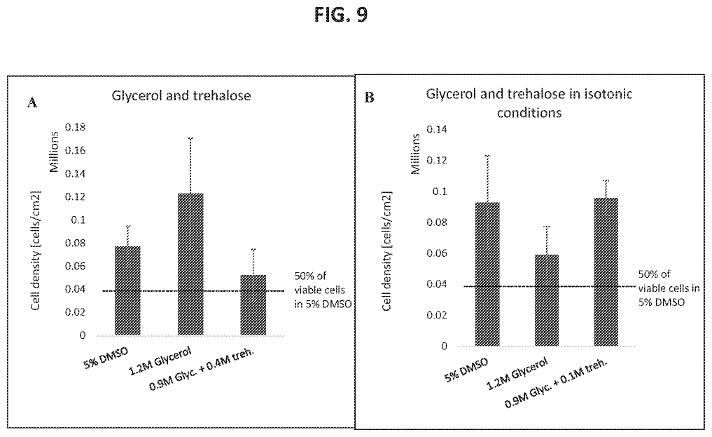

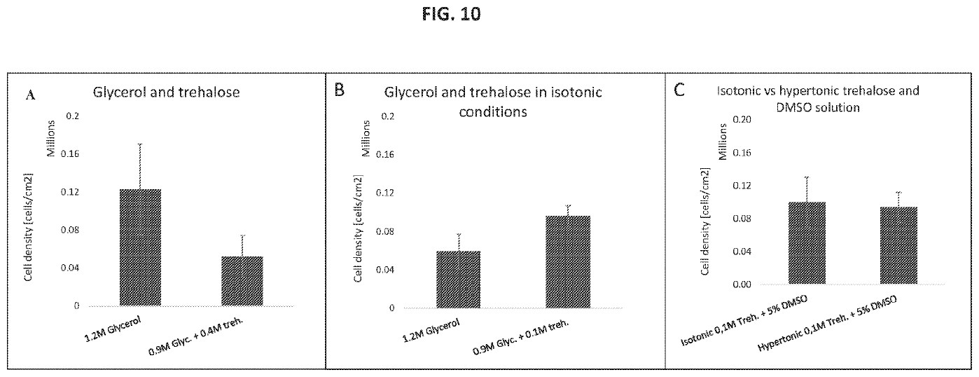

The results in FIG. 9A demonstrated that a 0.4M hypertonic solution of trehalose with 0.9M glycerol provided a decrease in viability compared to 1.2M glycerol formulation. In subsequent experiment it was then determined that the impairing factor was indeed the increased tonicity. As it is illustrated in FIG. 9B, the post-thaw cell viability increased when a lower tonicity was used, relative to 1.2M glycerol formulation. Non-superiority of hypertonic condition was furthermore demonstrated in the experiment, where the same concentrations of trehalose and DMSO were compared in different tonicities. Isotonic solution of trehalose was prepared by diluting the PBS in which the formulation was prepared. The hypertonic formulation was prepared with undiluted PBS. The results, presented in FIG. 10, does not show any significant difference between the two.

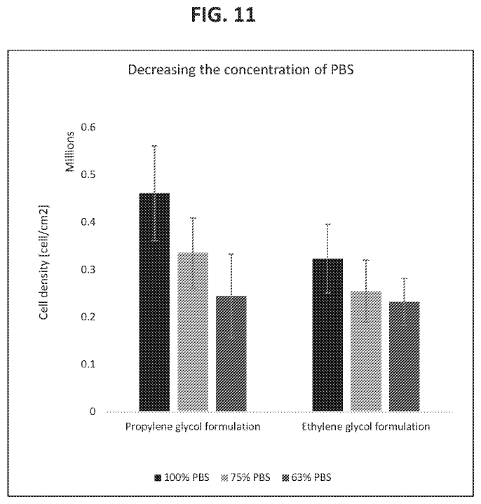

The negative impact of lowering the tonicity of cryopreservative solution to hypotonic conditions was demonstrated in the experiment, where the cell densities of plated cells after freeze-thawing were compared in different PBS concentrations. FIG. 11 shows the decrease in cell viability after decreasing the concentration of PBS. The principle was proved in two polyol formulations—with propylene glycol and ethylene glycol. This data suggests that hypertonic conditions do not show any improvement of cell survival during freeze-thawing and that isotonic conditions are preferred over hypotonic.

### Medium Supplements

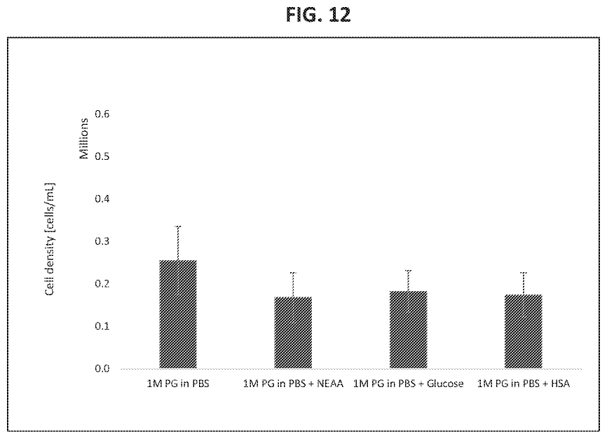

The cell medium includes substances that are vital for cell survival on longer term. Thus, Applicants thought that including some established cell medium ingredients into our formulations would increase the cell survival of the freeze-thawing process. For this purpose, the additions of non-essential amino acids (NEAA), human serum albumin (HSA) and glucose into the formulations were tested. Supplements were added to 1M propylene glycol formulation and compared against the solution of 1M propylene glycol. The results, which are illustrated in FIG. 12, prove that the additions of NEAA, HSA and glucose have no major benefit on cell survival during the freeze-thaw process.

The concentrations of supplements inside solutions were 2% (v/v) NEAA, 4.5 g/L glucose and 2.5% HSA.

### Thermal Treatment for Introduction of Trehalose Into Cells

In order to make cryo- and lyoprotection more efficient, a method for internalizing the excipients into cells was examined. In this way, the excipients (trehalose) would not only protect the cells extracellularly, but also intracellularly. If cells survived the treatment of internalization of excipients, subsequent freeze-thaw and then to freeze drying attempts would follow.

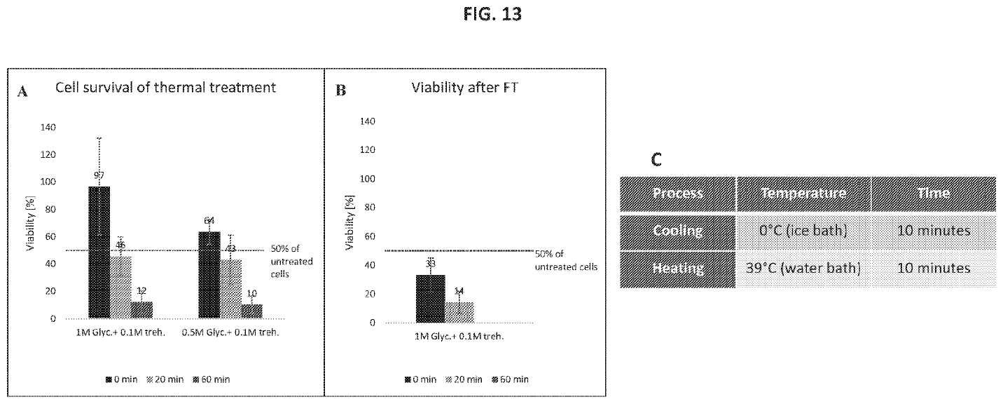

In order for the internalization of trehalose to be efficient, the cells have to first survive the thermal treatment. For this reason, the cell viability was tested before commencing with first step of cooling the cells and next after 20 minutes as well as after 60 minutes of thermal treatment. FIG. 13A shows that cells were dying at high rates with time of thermal treatment. The cells, which survived the treatment, also did not show an improved survival of the freeze-thawing. In FIG. 13B it can be seen, that a very low quantity of cells was recovered after freeze thawing process in 1M glycerol and 0.5M trehalose formulation. There was no cell survival in formulation with 0.5M glycerol and 0.1M trehalose. FIG. 13C describes the conditions of the freeze/thaw cycle.

### 2D and 3D Attached Cells

Further experiments were pointed towards utilizing attached cells instead of suspended ones, in order to decrease the handling stress on the cells prior freezing. Another hypothesis was that a hydrogel would provide additional protection for cells together with a bulkier lyophilization cake, that is easy to reconstitute and biocompatible. 2D attachment on collagen coated glass vials and 3D attachment on hyaluronan hydrogel matrix was tested for this purpose. After successfully culturing the cells on the surface of vials and in hydrogel matrix, we proceeded to freeze-thaw and freeze-drying experiments.

### Cell Culturing

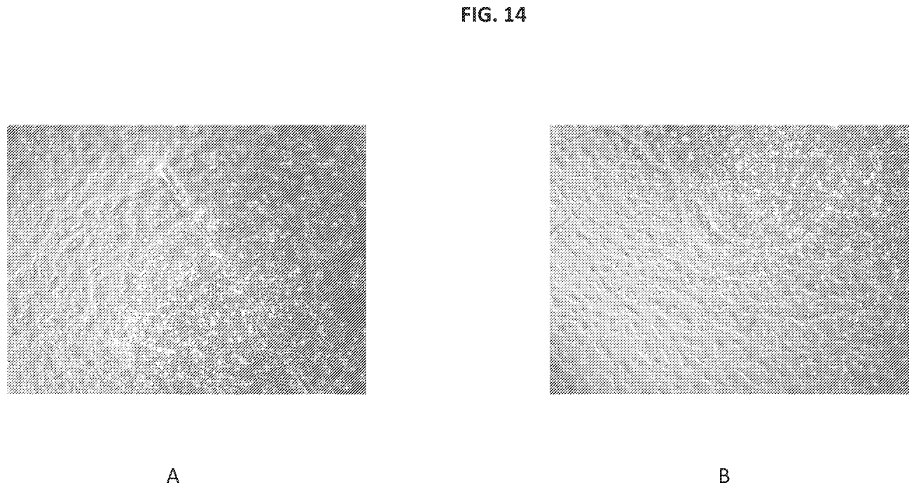

Two protocols for collagen coating of glass vials were tested and cells were subsequently seeded on both. Cell proliferation after one day of incubation in both conditions is presented in FIG. 14. Since both protocols provided a good cell attachment and proliferation, protocol A, as described above, was used for future coating, because it prescribes optimal amount of collagen. For 3D cell culture the cells were firstly seeded in TRANSWELL® inserts of commercial HYSTEM-HP® and 10 mg/mL hyaluronic acid (HA), respectively. HYSTEM-HP® has in addition to crosslinked hyaluronic acid molecules, also thiol-modified denatured collagen, which provides the hydrogel with more attachment sites for cells. Contrary to the expected high attachment and proliferation of cells in HYSTEM-HP®, there was no cell growth observed time. In HA the growth was better, however the cell morphology was similar as in 2D cultures (FIG. 14). This is probably because the HA solution used was not crosslinked and viscous enough. On dilution of the hydrogel with medium, cells easily settled on the surface of membrane of TRANSWELL® inserts and grow probably in 2D on the membrane.

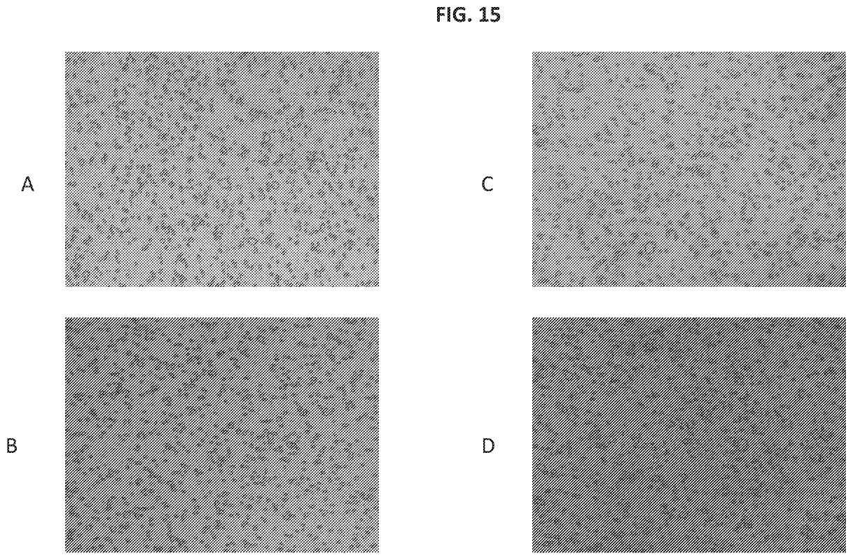

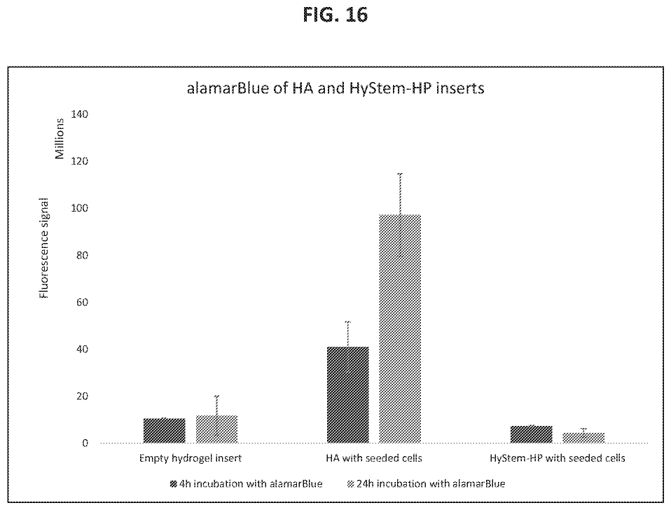

The high cell growth was further confirmed by ALAMARBLUE® measurements presented in FIG. 15. The metabolic reagent was incubated for 24 hours with the cells and fluorescence was measured after 4 h and 24 h of incubation. The increase in fluorescence signal in HA inserts between 4 h and 24 h is due to the higher quantity of metabolized resazurin, which gives out fluorescence. This confirms that the cells were indeed viable in HA gel, in contrary to HYSTEM-HP® inserts, where there was no increase in signal after 4 and 24 h of incubation with ALAMARBLUE®. The fluorescence of these inserts also did not differ from the blank. Latter was the HYSTEM-C® scaffold, which is similar to HYSTEM-HP® (FIG. 16) and was not seeded with cells.

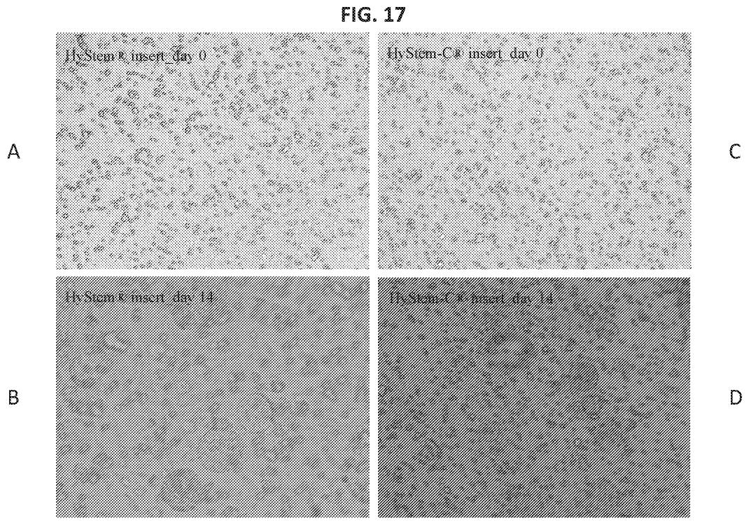

Another set of cells grown in 3D hydrogel scaffolds were prepared in HYSTEM® and HYSTEM-C®. Differences between these hydrogels are illustrated in FIG. 17. Cells suspended in hydrogel scaffolds were prepared both in TRANSWELL® inserts and inside 6R vials. By monitoring the growth under the optical microscope, it was possible to observe higher growth in the inserts, than in vials. This is likely, due to the better exchange and penetration of medium in the inserts. 3D cell growth manifested itself in sphere-like cell structures (FIG. 17). More spheres were viewed in HYSTEM-C® scaffold than in HYSTEM®, when the hydrogel was placed in TRANSWELL® inserts. This is may be the consequence of more attachment sites, that thiolated collagen provides in HYSTEM-C® type of hydrogel.

### Freeze/Thaw of the Attached Cells

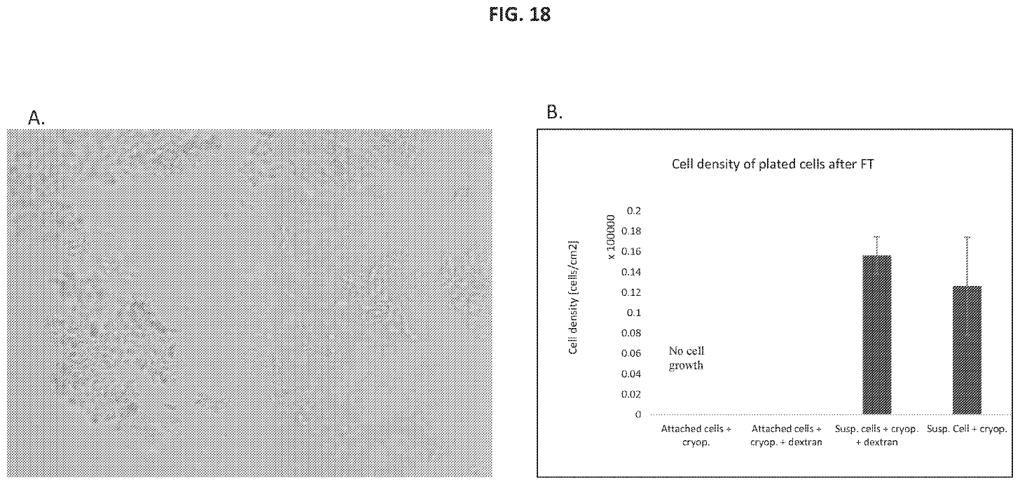

First the results of cell survival of freeze-thawed samples in 2D were evaluated. After culturing inside the vials, the cells were counted and fresh cells suspended in the same concentration in a cryopreservative solution (1M glycerol with 0.1M trehalose) for comparison of the results. After Freeze/Thaw, it was noticed under the microscope, that the 2D cultured cells were not attached to the glass surface anymore and they were floating in larger aggregates in the solution. All the conditions were then plated in a 96-well plate and after overnight incubation the ALAMARBLUE® assay was performed. The 2D cultured cells did not show any cell growth, contrary to the cells that were suspended in the solution, as it can be seen in FIG. 18B.

The reason for cell death may be due to the process of quick thawing from −80° C. to 37° C., as it probably can cause the glass and collagen coating to expand very rapidly and unevenly. This may have caused the cracks in collagen and in cell layer, which then could lead to cell detachment and death. Cell detachment was clearly observed under the microscope, which can be seen in FIG. 18A. Similar observation was reported by Campbell and Brockbank, who concluded that rapid thawing caused shape changes of plastic of 96-well plate, in which they were culturing the cells. See, Recent Advances in Cryopreservation (2014). doi:10.5772/58618. In their opinion, this caused forceful cell detachment and death.

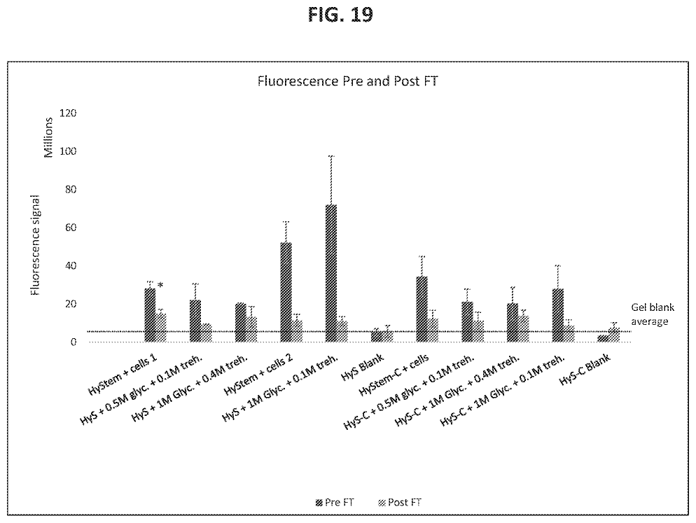

Next, the Freeze/Thaw was performed on the 3D grown cell cultures in TRANSWELL® inserts. The cell density varied between respective inserts, as can be seen in FIG. 19. After thawing the fluorescence decreased considerably in all samples, the ALAMARBLUE® assay was performed for each sample in triplicates and the results showed a large variance, most likely due to the difficulty of homogenizing the sample properly before fluorescence measurement. Gels without cells are not able to reduce resazurin to fluorescent form and were chosen as a blank. Since empty gels have similar properties, they produced similar results. Hence, these results were grouped together to calculate the average blank signal. This calculation was used for t-test where, the results of post Freeze/Thaw samples were compared with the average blank signal. If a fluorescence signal of a sample is significantly different from a blank, it indicates the survival of cells. The conditions that proved to preserve a certain amount of cells over the treatment are: HYSTEM® with no cryoprotectant, HYSTEM®with 0.5M glycerol and O.1M trehalose & HYSTEM-C® with 1M glycerol and 0.4M trehalose. The results of this experiment are preliminary and were performed on one sample. Hence, further investigation of cryoprotective ability of these combinations is necessary. The fluorescence of each sample was measured three times and error bars in the FIG. 19 show the standard deviation between measurements. Latter is relatively high in some cases, due to difficulty to homogenize the samples with hydrogel scaffolds.

### Freeze Drying Experiments

The best formulations, which allowed cells to withstand freeze-thawing were tested for their ability to be lyophilized. From literature it is known, that for certain cell lines, the cells start to die during secondary drying with an increased rate. Thus, a hypothesis was made, that in order to extend the shelf life of the cell products, water is not required to be completely removed from formulation. Hence, only conservative primary drying cycles were employed in the process and no secondary drying, as described above.

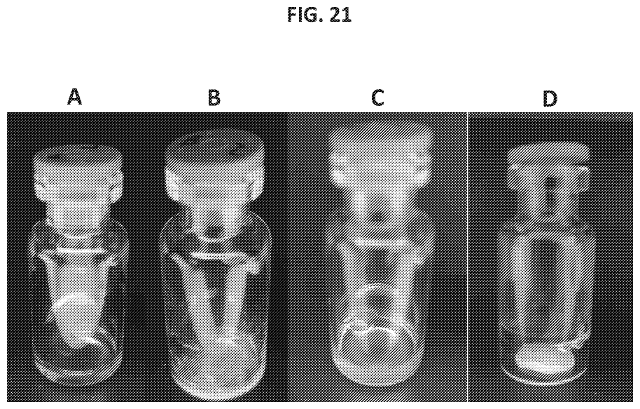

Visual inspection of various 2D and 3D formulations was performed. FT results showed that the best cell viabilities were achieved using glycerol/trehalose and propylene glycol/trehalose combinations. These formulations were freeze dried and FIG. 21 shows the resulting lyophilizates. 1M glycerol with 0.1M trehalose formulation did not produce any cake, which means that the formulation by itself is not lyophilizable. When the glycerol content was decreased to half, a gel-like structure was obtained after lyophilization. This improvement was due to the lower decrease in Tg′, which is caused by high amount of polyols. In contrast to glycerol, propylene glycol did not undergo sublimation and remained in liquid form after the process. Addition of 10 mg/mL dextran into 1M glycerol and 0.1M trehalose formulation, showed an improvement in the appearance of the product. The reason for this is likely to be a higher amount bulking excipient—dextran and the fact, that this excipient also increases T_{g}′. A substantial shrinkage was still observed in the lyophilizate, but on the other hand the impeccable elegance is not the primary objective and its relation to cell viability is not yet established.

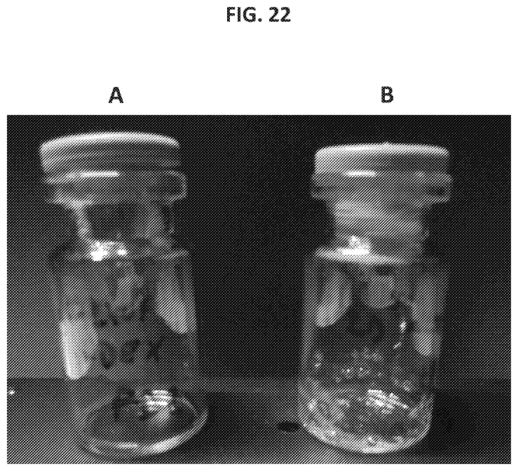

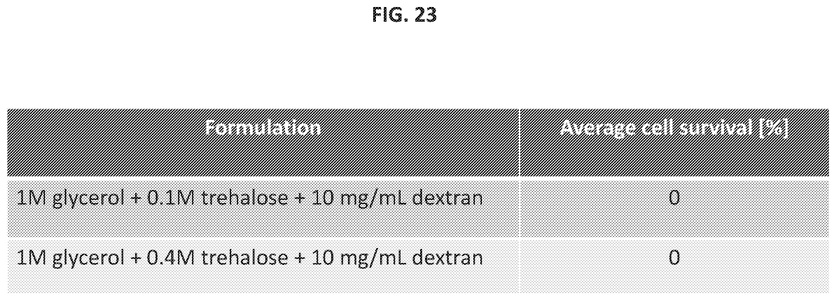

Visual inspection of 2D formulations with a population of cells in (A) 1M glycerol+0.1M trehalose+10 mg/mL dextran, and (B) 1M glycerol+0.4 M Trehalose+10 mg/mL dextran (0.5 mL volume). See FIG. 22. Neither formulation provided a lyophilized cake structure. FIG. 23 shows the cell survivability of the lyophilized formulations of FIG. 22. None of the formulations provided viable cells.

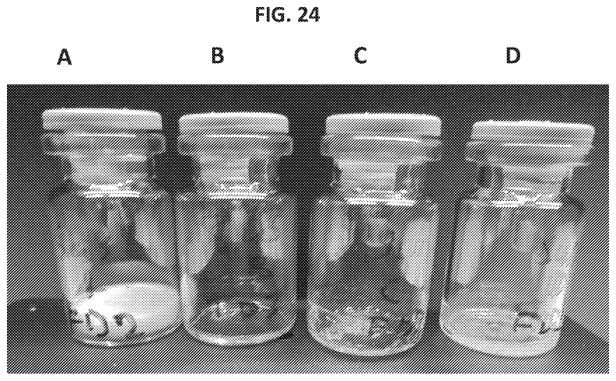

The FIG. 24 shows visual inspection of formulation with suspended cells in (A) hyaluronic acid (15 mg/mL), (B) 1M glycerol+0.1M trehalose+10 mg/mL dextran, (C) 1M glycerol+0.4 M trehalose, and (D) 1M glycerol+0.4M trehalose+10 mg/mL dextran (0.5 mL volume). Hyaluronic acid provided a lyophilized cake structure (FIG. 24A). Addition of polyol (glycerol) and sugar (trehalose) showed a collapse of the cake structure (FIG. 24B-D). However, higher concentration of trehalose in combination with dextran showed an improvement in the cake structure (FIG. 24D).

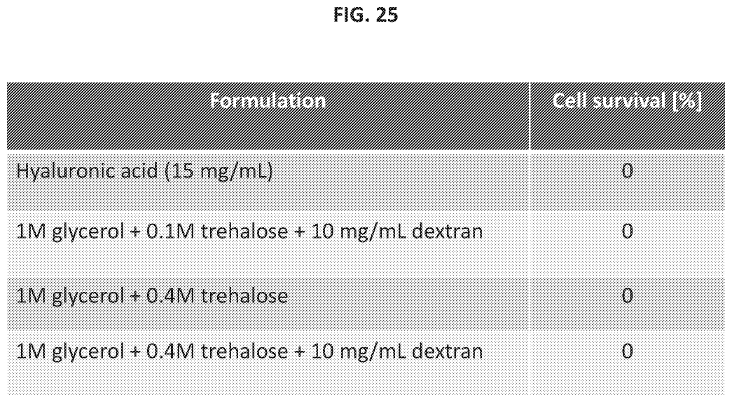

FIG. 25 shows the cell survivability of the lyophilized formulations of FIG. 24. None of the formulations provided viable cells.

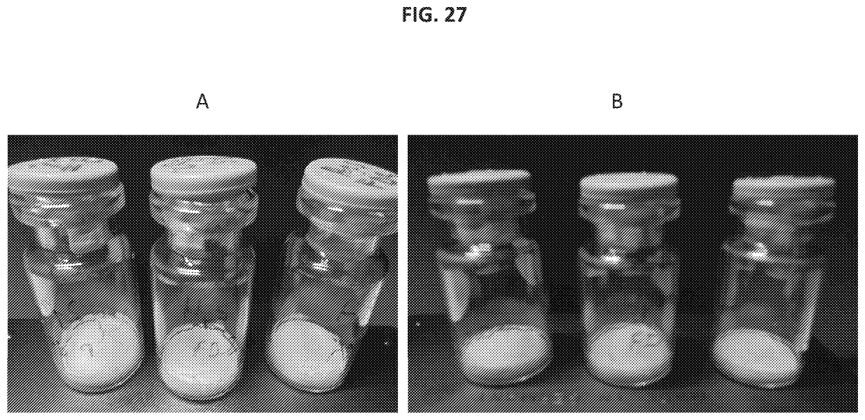

FIG. 27 shows visual inspection of a population of cells suspended in (A) HYSTEM® hydrogel and (B) HYSTEM-C® hydrogel. Both formulations provided a lyophilized cake structure

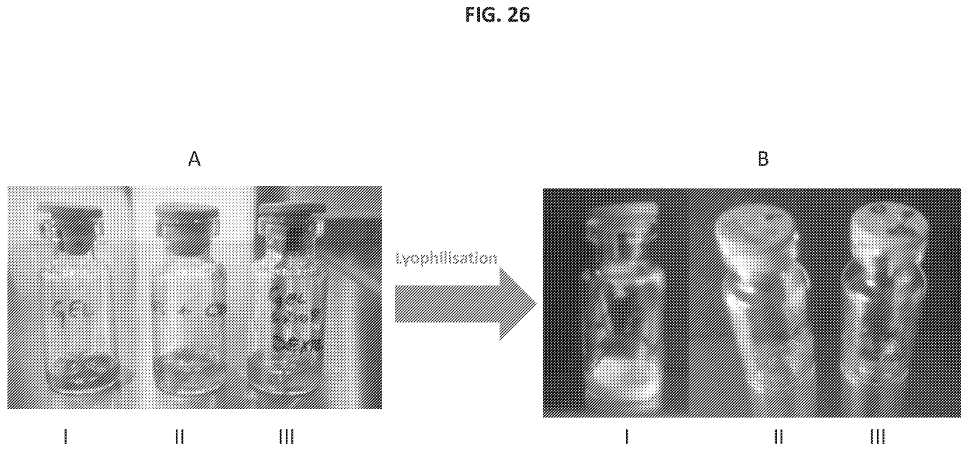

FIG. 26 shows visual inspection of a population of cells suspended in (I) HyStem-HP hydrogel, (II) HyStem-HP hydrogel+1M glycerol+0.1 M trehalose, and (III) HyStem-HP hydrogel+1M glycerol+0.1 M trehalose+10 mg/mL dextran. FIG. 26 provides visualization for the formulations comprising the cells before (panel A) and after (panel B) lyophilization. The addition of a cryopreservative, polyol containing solution induced the collapse of the cake structure. 10 mg/mL of dextran, which improved the appearance of lyophilizates with non-attached cell formulations, did not show any improvement in this case. This collapse of the structure could potentially be avoided by reducing the amount of cryopreservant or preparation of scaffold with cryopreservative solution instead of degassed water.

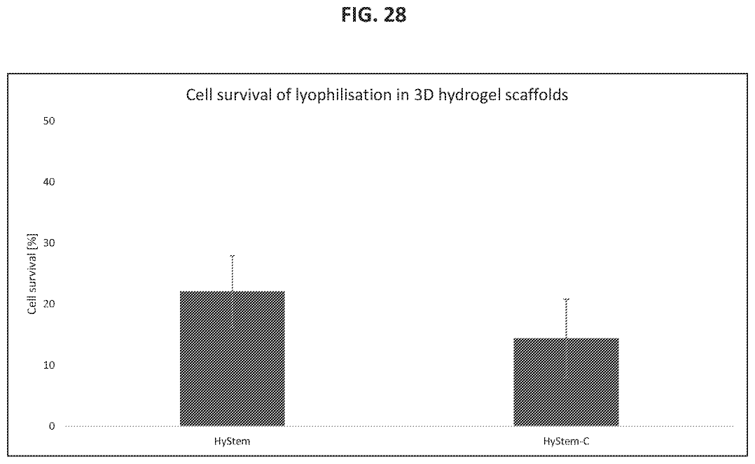

The viability of the cells in the absence of cryoprotectant was investigated. FIG. 28 shows the cell viability of 3D formulations with suspended population of cells in (A) HyStem and (B) HyStem-C without any cryoprotectants. Cells in both HyStem formulations without cryoprotectant both exhibited >15-20% cell viability.

### Conclusion

Due to logistical impracticality and price drawbacks of cryopreservation (frozen storage of cells), an alternative approach of freeze drying of cell therapy product model line (SK-N-AS) was studied. For designing a formulation for such a lyophilizate, we firstly screened for alternatives to commonly used DMSO, which is poorly lyophilizable and brings up toxicity concerns. Three polyols have emerged as potential replacements. While ethylene glycol showed a lower performance as a cryoprotectant, literature also suggest toxicology concerns. Propylene glycol and glycerol were shown as viable alternatives for cryopreservation using DMSO, however propylene glycol proved to be not favorable for lyophilization at tested lyophilization conditions. Glycerol formulation was also poorly lyophilized when only 0.1M trehalose was added, but the addition of high concentration (10 mg/mL) of dextran showed major improvement. Also, cells were successfully cultured in 2D cell culture on glass vial surface and in 3D in hydrogel scaffold. 2D cultured cells with added cryoprotectants did not survive freeze-thawing process probably due to rapid thawing. Hydrogel scaffold provided a lyophilization cake appearance which was further compromised with cryopreservative addition.

### Example 6

The freeze-drying method according to Example 5 is performed with mesenchymal stem cells (MSCs). The MSCs are cultured in a 2D cell culture in a glass vial, a 3D cell culture in a hydrogel, or a 3D cell culture in a glass vial as described in Example 4. The MSCs are frozen in aqueous compositions containing dextran, a polyol, and optionally trehalose according to Tables 5-7.

TABLE 5
Trehalose concentrations in compositions for freezing MSCs.
Trehalose Concentration
0M
0.1M
0.2M
0.3M
0.4M
0.5M
0.6M
0.7M
0.8M
0.9M
1M

TABLE 6
Dextran concentrations in compositions for freezing MSCs.
Dextran Concentration

| | |
|---|---|
| 1 | mg/mL |
| 2 | mg/mL |
| 5 | mg/mL |
| 10 | mg/mL |
| 25 | mg/mL |

TABLE 7
Polyols included in compositions for freezing MSCs.

| Polyol | Concentration |
|---|---|
| Glycerol | 0.05M |
| | 0.1M |
| | 0.2M |
| | 0.5M |
| | 0.9M |
| | 1M |
| | 1.2M |
| | 1.5M |
| | 2M |
| | 3M |
| | 4M |
| | 5M |
| Ethylene Glycol | 0.05M |
| | 0.1M |
| | 0.2M |
| | 0.5M |
| | 0.9M |
| | 1M |
| | 1.2M |
| | 1.5M |
| | 2M |
| | 3M |
| | 4M |
| | 5M |
| Propylene Glycol | 0.05M |
| | 0.1M |
| | 0.2M |
| | 0.5M |
| | 0.9M |
| | 1M |
| | 1.2M |
| | 1.5M |
| | 2M |
| | 3M |
| | 4M |
| | 5M |

The freeze-dryer programs shown in Table 3 (for MSC suspensions) and Table 4 (for MSC hydrogels) are employed for freeze-drying the MSCs. Viability of the lyophilized MSCs is evaluated using visual inspection of the lyophilized cake structure, ALAMARBLUE® assay, or both.

### Example 7

The freeze-drying method according to Example 5 is performed with induced pluripotent stem cells (iPSCs). The iPSCs are cultured in a 2D cell culture in a glass vial, a 3D cell culture in a hydrogel, or a 3D cell culture in a glass vial as described in Example 4. The iPSCs are frozen in: aqueous compositions containing dextran, a polyol, and optionally trehalose according to Tables 5-7.

The freeze-dryer programs shown in Table 3 (for iPSC suspensions) and Table 4 (for iPSC hydrogels) are employed for freeze-drying the iPSCs. Viability of the lyophilized iPSCs is evaluated using visual inspection of the lyophilized cake structure, ALAMARBLUE® assay, or both.

All references cited herein, including patents, patent applications, papers, textbooks and the like, and the references cited therein, to the extent that they are not already, are hereby incorporated herein by reference in their entirety.

What is claimed is:

1. A method of producing a population of lyophilized cells, comprising:
(a) freezing a composition comprising a population of cells, an aqueous component, a polyol, a sugar, and a polysaccharide; and
(b) removing at least about 90% of the aqueous component from the frozen composition to produce the population of lyophilized cells.

2. A method of producing a population of reconstituted viable cells, comprising:
(a) freezing a composition comprising a population of cells, an aqueous component, a polyol, a sugar, and a polysaccharide;
(b) removing at least about 90% of the aqueous component from the frozen composition to produce the population of lyophilized cells, and
(c) resuspending the population of lyophilized cells in a reconstitution agent to form a reconstituted composition, wherein at least about 1% of the cells are viable.

3. The method of claim 1 or 2, wherein the composition of (a) further comprises a hydrogel.

4. The method of claim 3, wherein the hydrogel is a biocompatible hydrogel.

5. The method of claim 3, wherein the hydrogel is a hyaluronan gel, alginate gel, agarose gel, collagen gel or combination of thereof.

6. The method of any one of claims 1 to 5, wherein the population of cells in (a) is suspended in the hydrogel.

7. The method of any one of claims 1 to 6, wherein the freezing of (a) is performed either in suspension or attached in a container.

8. The method of any one of claims 1 to 6, wherein the freezing of (a) is performed with the population of cells suspended in a hydrogel.

9. The method of any one of claims 1 to 8, wherein the lyophilization is performed in a container with or without collagen coating.

10. The method of any one of claims 1 to 9, wherein the container is a glass or plastic container without or without membrane for cell attachment or growth.

11. The method of any one of claims 1 to 10, wherein the population of cells of (a) is about 1×10⁴ cells per mL to 1×10⁶ cells per mL.

12. The method of any one of claims 1 to 11, wherein the population of cells of (a) is about 1×10⁵ cells per mL to 4×10⁵ cells per mL.

13. The method of any one of claims 1 to 12, wherein the population of cells of (a) is about 2×10⁵ cells per mL to 2.5×10⁵ cells per mL.

14. The method of any one of claims 1 to 13, wherein the sugar is a disaccharide.

15. The method of claim 14, wherein the disaccharide is selected from the group consisting of maltose, lactose, sucrose, lactulose, trehalose, cellobiose, isomaltose, melibiose, and gentiobiose.

16. The method of claim 14 or 15, wherein the disaccharide has a Ci-Ci glycosidic linkage.

17. The method of any one of claims 1 to 13, wherein the sugar is trehalose.

18. The method of any one of claims 1 to 17, wherein prior to the freezing in (a), the population of cells is isolated and resuspended in a poration solution.

19. The method of claim 18, wherein the poration solution comprises sugar and water.

20. The method of claim 19, wherein the sugar in the poration solution is about 0.1 M to about 1.0M.

21. The method of any one of claims 18 to 20, wherein the poration solution comprises trehalose and water.

22. The method of claim 21, wherein the trehalose in the poration solution is about 0.1 M to about 1.0M.

23. The method of claim 21 or 22, wherein the trehalose in the poration solution is about 0.2 M to about 0.6 M.

24. The method of any one of claims 21 to 23, wherein the trehalose in the poration solution is about 0.4 M.

25. The method of any one of claims 1 to 24, wherein the polyol is selected from glycerol, propylene glycol and ethylene glycol.

26. The method of any one of claims 1 to 25, wherein the polyol is about 0.05 M to about 5 M.

27. The method of any one of claims 1 to 26, wherein the polyol is about 0.1 M to about 3 M.

28. The method of any one of claims 1 to 27, wherein the polyol is about 0.5 M to about 2 M.

29. The method of any one of claims 1 to 28, wherein the polyol is about 1 M to about 1.5 M.

30. The method of any one of claims 1 to 29, wherein the polyol is glycerol.

31. The method of claim 30, wherein the glycerol is about 0.01 M to about 10 M in the composition.

32. The method of claim 30, wherein the glycerol is about 0.05 M to about 5 M.

33. The method of claim 30, wherein the glycerol is about 0.1 M to about 3 M.

34. The method of claim 30, wherein the glycerol is about 0.5 M to about 2 M.

35. The method of claim 30, wherein the glycerol is about 1 M to about 1.5 M.

36. The method of any one of claims 1 to 35, wherein the polysaccharide is about 1 mg/mL to about 25 mg/mL.

37. The method of any one of claims 1 to 36, wherein the polysaccharide is about 5 mg/mL to about 15 mg/mL.

38. The method of any one of claims 1 to 37, wherein the polysaccharide is about 10 mg/mL.

39. The method of any one of claims 1 to 38, wherein the polysaccharide is a polyglucan.

40. The method of claim 39, wherein the polyglucan is a dextran.

41. The method of claim 40, wherein the dextran has a molecular weight of about 10,000 Da to about 60,000 Da.

42. The method of claim 40, wherein the dextran has a molecular weight of about 30,000 Da to about 50,000 Da.

43. The method of claim 40, wherein the dextran has a molecular weight of about 40,000 Da.

44. The method of any one of claims 1 to 43, wherein the composition of (a) is free of DMSO.

45. The method of any one of claims 1 to 44, wherein the composition comprising the population of cells in (a) comprises thiobarbituric acid, polyvinylpyrrolidone, polyoxyethylene stearyl ether, polyethylene glycol, cyclodextrin, hydroxyethyl starch, or combinations thereof.

46. The method of any one of claims 2 to 45, wherein the reconstitution agent comprises polyvinylpyrrolidone, trehalose, sucrose, glucose or combinations thereof.

47. The method of any one of claims 2 to 46, wherein the reconstitution agent comprises polyvinylpyrrolidone.

48. The method of any one of claims 2 to 47, wherein the reconstitution agent comprises a phosphate buffer solution.

49. The method of any one of claims 1 to 48, wherein the composition is an isotonic solution.

50. The method of any one of claims 1 to 49, wherein the composition is a hypertonic solution.

51. The method of any one of claims 1 to 50, wherein the cell is a mammalian cell.

52. The method of any one of claims 1 to 51, wherein the cell is a stem cell.

53. The method of any one of claims 1 to 52, wherein the cell is a pluripotent stem cell, an embryonic stem cell, a mesenchymal stem cell, or a hematopoietic stem cell.

54. The method of any one of claims 1 to 53, wherein the cell is a mesenchymal stem cell.

55. The method of any one of claims 1 to 54, wherein the cell is an induced pluripotent stem cell.

56. The method of any one of claims 1 to 51, wherein the population of cells are neuroblastoma cells.

57. The method of claim 56, wherein the neuroblastoma cells are SK-N-AS cells.

58. The method of any one of claims 1 to 57, wherein the aqueous component comprises buffer.

59. The method of any one of claims 1 to 58, wherein the buffer comprises phosphate buffer, Tris buffer, acetate buffer, bicarbonate buffer, histidine buffer, citrate buffer, or combinations thereof.

60. The method of any one of claims 1 to 59, wherein the aqueous component comprises cell culture medium.

61. The method of claim 60, wherein the cell culture medium is free of serum.

62. The method of any one of claims 1 to 61, wherein the removing the aqueous component comprises lowering pressure, applying heat, or both, to the frozen composition to remove the aqueous component.

63. The method of any one of claims 1 to 62, wherein the removing the aqueous component comprises a primary drying step and a secondary drying step.

64. The method of any one of claims 1 to 63, wherein the removing the aqueous component comprises greater than two drying steps.

65. The method of any one of claims 1 to 64, wherein the primary drying step comprises lowering pressure to remove the aqueous component.

66. The method of any one of claims 1 to 65, wherein the secondary drying step comprises applying heat to remove the aqueous component.

67. The method of any one of claims 1 to 66, wherein the freezing occurs at between about −10° C. to about −100° C.

68. The method of any one of claims 1 to 67, wherein the freezing occurs at between about −20° C. to about −90° C.

69. The method of any one of claims 1 to 68, wherein the freezing occurs at between about −40° C. to about −60° C.

70. The method of any one of claims 1 to 69, wherein the freezing of (a) lowers the temperature of the composition to about −50° C.

71. The method of any one of claims 1 to 70, wherein the freezing of (a) lowers the temperature of the composition to about −50° C., the aqueous component is removed at a pressure of a chamber pressure of about 20 mTorr to about 40 mTorr.

72. The method of any one of claims 2 to 71, wherein the resuspending the lyophilized cell occurs greater than two hours after the removing the aqueous component.

73. The method of any one of claims 2 to 72, wherein the resuspending the lyophilized cells occurs greater than one day after the removing the aqueous component.

74. The method of any one of claims 2 to 73, wherein the resuspending the lyophilized cells occurs greater than one week after the removing the aqueous component.

75. The method of any one of claims 2 to 74, wherein the resuspending the lyophilized cells occurs greater than one month after the removing the aqueous component.

76. The method of any one of claims 1 to 75, wherein the lyophilized cells are stored below about −20° C. prior to the resuspending.

77. The method of any one of claims 1 to 75, wherein the lyophilized cells are stored at about −20° C. to about 30° C. prior to the resuspending.

78. The method of any one of claims 1 to 75, wherein the lyophilized cells are stored at about 4° C. to about 28° C. prior to the resuspending.

79. The method of any one of claims 1 to 75, wherein the lyophilized cells are stored at about 10° C. to about 27° C. prior to the resuspending.

80. The method of any one of claims 1 to 75, wherein the lyophilized cells are stored at about 15° C. to about 26° C. prior to the resuspending.

81. The method of any one of claims 1 to 75, wherein the lyophilized cells are stored at about 20° C. to about 25° C. prior to the resuspending.

82. The method of any one of claims 1 to 75, wherein the lyophilized cells are stored at about 2° C. to about 8° C. prior to the resuspending.

83. The method of any one of claims 1 to 82, wherein the lyophilized cells are stored at greater than about −20° C. for greater than 2 days prior to the resuspending.

84. The method of any one of claims 1 to 82, wherein the lyophilized cells are stored at about 20° C. to about 25° C. for greater than 1 week prior to the resuspending.

85. The method of any one of claims 1 to 82, wherein the lyophilized cells are stored at about 2° C. to about 8° C. for greater than 1 week prior to the resuspending.

86. The method of any one of claims 1 to 85, wherein at least about 20% of the cells in the reconstituted composition are viable.

87. The method of any one of claims 1 to 86, wherein at least about 30% of the cells in the reconstituted composition are viable.

88. The method of any one of claims 1 to 87, wherein at least about 40% of the cells in the reconstituted composition are viable.

89. The method of any one of claims 1 to 88, wherein at least about 50% of the cells in the reconstituted composition attach as measured by ALAMARBLUE test.

90. A composition comprising a population of cells, an aqueous component, a polyol, a sugar, and polysaccharide, wherein the composition is less than about 10% (vol/vol) water, and wherein the population of cells can be resuspended, wherein at least about 1% the cells in the population of cells are viable when resuspended.

91. A composition comprising a population of cells, an aqueous component, glycerol, trehalose, and polysaccharide, wherein the composition is less than about 10% (vol/vol) water, and wherein the population of cells can be resuspended, wherein at least about 1% the cells in the population of cells are viable when resuspended.

92. A composition comprising a population of cells, an aqueous component, about 0.5 M to about 2 M polyol, about 0.2 M to about 0.6 M sugar, and about 2 mg/mL to about 20 mg/mL polysaccharide, wherein the population of cells is about 2×10⁵ cell/cm² to about 3×10⁵ cells/cm².

93. A composition comprising a population of cells, an aqueous component, about 0.5 M to about 2 M glycerol, about 0.2 M to about 0.6 M trehalose, and about 2 mg/mL to about 20 mg/mL polysaccharide, wherein the population of cells are about 2×10⁵ cell/cm² to about 3×10⁵ cells/cm².

94. The composition of claim 93, wherein the aqueous component in the population of cells is less than about 5% w/w.

95. A method of lyophilizing a population of cells, comprising:
(a) freezing a composition comprising the population of cells, an aqueous component, about 0.5 M to about 2 M glycerol, about 0.2 M to about 0.6 M trehalose, and about 2 mg/mL to about 20 mg/mL polysaccharide, wherein the freezing occurs below about −30° C.; and
(b) removing the aqueous component from the frozen composition to produce a population of lyophilized cells, wherein the aqueous component in the population of lyophilized cells is less than about 5% w/w.

96. The method of claim 95, wherein the composition of (a) further comprises a hydrogel.

97. The method of claim 96, wherein the hydrogel is a hyaluronan gel, alginate gel or collagen gel.

98. The method of any one of claims 95 to 97, wherein the population of cells in (a) is suspended in the hydrogel.

99. A method of producing viable population of cells, comprising:
(a) freezing a composition comprising the population of cells, an aqueous component, about 0.5 M to about 2 M polyol, about 0.2 M to about 0.6 M sugar, and about 2 mg/mL to about 20 mg/mL polysaccharide, wherein the freezing occurs below about −30° C.;
(b) removing the aqueous component from the frozen composition to produce a population of lyophilized cells, wherein the aqueous component in the population of lyophilized cells is less than about 5% w/w; and
(c) resuspending the population of lyophilized cells in a reconstitution agent to form a reconstituted composition, wherein at least about 5% of the cells in the reconstituted composition are viable.

100. A method of producing viable population of cells, comprising:
(a) freezing a composition comprising the population of cells, an aqueous component, about 0.5 M to about 2 M glycerol, about 0.2 M to about 0.6 M trehalose, and about 2 mg/mL to about 20 mg/mL polysaccharide, wherein the freezing occurs below about −30° C.;
(b) removing the aqueous component from the frozen composition to produce a population of lyophilized cells, wherein the aqueous component in the population of lyophilized cells is less than about 5% w/w; and
(c) resuspending the population of lyophilized cells in a reconstitution agent to form a reconstituted composition, wherein at least about 1% of the cells in the reconstituted composition are viable.

101. The method of any one of claim 99 or 100, wherein the composition of (a) further comprises a hydrogel.

102. The method of claim 101, wherein the hydrogel is a hyaluronan gel, alginate gel or collagen gel.

103. The method of claim 102, wherein the population of cells in (a) is suspended in the hydrogel.

104. A method of producing a population of lyophilized cells, comprising:
(a) freezing a composition comprising a population of cells and an aqueous component in a hydrogel matrix; and
(b) removing at least about 90% of the aqueous component from the frozen composition to produce the population of lyophilized cells.

105. A method of producing a population of reconstituted viable cells, comprising:
(a) freezing a composition comprising a population of cells and an aqueous component in a hydrogel matrix;
(b) removing at least about 90% of the aqueous component from the frozen composition to produce the population of lyophilized cells, and
(c) resuspending the population of lyophilized cells in a reconstitution agent to form a reconstituted composition, wherein at least about 1% of the cells are viable.

106. The method of claim 104 or 105, wherein the hydrogel is a biocompatible hydrogel.

107. The method of any one of claims 104 to 106, wherein the hydrogel is a hyaluronan gel, alginate gel or collagen gel.

108. The method of any one of claims 104 to 107, wherein the population of cells in (a) is suspended in the hydrogel.

109. The method of any one of claims 104 to 108, wherein the composition further comprises a polyol, a sugar, a polysaccharide, or combinations thereof.

110. The method of any one of claims 104 to 109, wherein the population of cells of (a) is about 1×10⁴ cells per mL to 1×10⁶ cells per mL.

111. The method of claim 109 or 110, wherein the sugar is a disaccharide.

112. The method of claim 111, wherein the disaccharide is selected from the group consisting of maltose, lactose, sucrose, lactulose, trehalose, cellobiose, isomaltose, melibiose, and gentiobiose.

113. The method of claim 109 or 110, wherein the sugar is trehalose.

114. The method of any one of claims 109 to 113, wherein the polyol is selected from glycerol, propylene glycol and ethylene glycol.

115. The method of any one of claims 109 to 114, wherein the polyol is about 0.05 M to about 5 M.

116. The method of any one of claims 109 to 115, wherein the polyol is glycerol.

117. The method of claim 116, wherein the glycerol is about 0.01 M to about 10 M in the composition.

118. The method of any one of claims 109 to 117, wherein the polysaccharide is about 1 mg/mL to about 25 mg/mL.

119. The method of any one of claims 109 to 118, wherein the polysaccharide is a polyglucan.

120. The method of claim 119, wherein the polyglucan is a dextran.

121. The method of claim 120, wherein the dextran has a molecular weight of about 10,000 Da to about 60,000 Da.

122. The method of any one of claims 104 to 121, wherein the composition of (a) is free of DMSO.
